(19) United States
(12) Patent Application Publication
(10) Pub. No.: US 2023/0384294 A1
(43) Pub. Date: Nov. 30, 2023

(54) METHOD OF DIAGNOSIS AND TREATMENT OF AUTISM SPECTRUM DISORDER

(71) Applicant: MOLECULAR YOU CORPORATION, Vancouver (CA)

(72) Inventors: Mohammad Ashraful ANWAR, Vancouver (CA); Zhengnan WANG, Vancouver (CA); Robert FRASER, Vancouver (CA); David WISHART, Vancouver (CA); Rupasri, Vancouver (CA)

(21) Appl. No.: 18/027,609

(22) PCT Filed: Sep. 21, 2021
(86) PCT No.: PCT/CA2021/051309

§ 371 Date: Mar. 21, 2023

Related U.S. Application Data
(60) Provisional application No. 63/080,842, filed on Sep. 21, 2020.
(60) Provisional application No. 63/115,221, filed on Nov. 18, 2020.

(51) Int. Cl. G01N 33/53; G01N 33/92

(52) U.S. Cl. CPC G01N 33/5308; G01N 33/92; G01N 2800/28; G01N 2800/52; G01N 2570/00

(57) ABSTRACT

The present disclosure relates to methods and kits for the diagnosis and treatment of autism spectrum disorder (ASD) in human subjects. The disclosure also relates to computer-implemented methods for diagnosing and treating ASD. Current diagnostic protocols are mainly limited to behavioural examination as laboratory findings have been consistently abnormal in ASD. No currently reported biomarker holds promise as early developmental screen or an early diagnostic or prognostic tool for pediatric settings at young ages from birth through early childhood when these clinical tools are most needed. With the present disclosure, metabolomic profiles, protein profiles and combinations thereof for ASD are identified in the subject having ASD.

### TECHNICAL FIELD

The present disclosure relates generally to the field of diagnosing and management/treatment of autism spectrum disorder (ASD).

### BACKGROUND

Autism Spectrum Disorder (ASD) (also referred to herein as autism) is a serious neuropsychiatric and neurodevelopmental disorder characterized by numerous symptoms. Typically, subjects suffer disturbances in social behaviors/interactions, deficits in communication (verbal and non-verbal) and difficulties engaging in age appropriate activities and interests. For example, subjects often exhibit a range of conditions having varying degrees of impaired social behavior, frustrated communication and language, and/or a narrow range of interests and activities that are both unique to the individual and carried out repetitively. These symptoms can be present and become apparent in early child development, i.e., during the first 5 years of life, and may cause clinically significant impairments in social, learning, occupational or other important areas of life later on.

According to the World Health Organization, 1 in 160 children around the world has ASD. However, despite its high prevalence, there is a lack of adequate and/or accurate ASD screening tools. As a result, there is often a delay in diagnosis and therapeutic interventions. Most children are still being diagnosed late (e.g., after 4 to 5 years of age), even though early signs of ASD can be observed in children as young as 2 years old. This delay is problematic because it has been shown that physicians can vastly improve outcomes for individuals, particularly children, with ASD through early diagnosis and referral for evidence-based behavioral therapy.

Evidence-based psychosocial interventions, such as behavioural treatment and parent skills training programmes, can reduce difficulties in communication and social behaviour, with a positive impact on well-being and quality of life for persons with ASD and their caregivers. Unfortunately, the first sign of ASD commonly recognized by pediatricians is a deficit in communication and language that does not manifest until 18 to 24 months of age. ASD is a global public health phenomenon that is distributed throughout all strata of the population, with costly and lifelong impact due to vulnerability to stigmatization and discrimination, limitations on productivity, and some measure of dependency requiring supervision, support and protection. Early identification and early intervention of autistic children are recognized by the World Health Organization (“WHO”) as two of the most critical factors for improving outcomes for individuals affected by ASD.

To date, the ability to properly diagnose individuals with ASD is insufficient and primarily based on qualitative observations by trained specialists. Current diagnostic protocols are mainly limited to behavioural examination as laboratory findings have been consistently abnormal in ASD. For example, the current screening tools for ASD in this age group include the Infant Toddler Checklist (“ITC”; and also known as the Communication and Symbolic Behaviour Scales and Development Profile) and the Modified Checklist for Autism in Toddlers (“M-CHAT”). The ITC may be used to identify developmental deficits in children ages 9 to 24 months, but has limited utility in distinguishing basic communication delays from overt ASD. The M-CHAT may be employed between 16 and 30 months. However, it requires a follow-up questionnaire for positive screens, which occurs in 10% of the children. The mean age of diagnosis for children with ASD is about 3 years, and approximately half of these can be false positives. Moreover, an appropriately qualified person is required to diagnose such an individual using these standard behavioral testing protocols/guidelines, which may also introduce a layer of subjectivity to the traditional assessment approach.

ASD is highly heterogeneous and its etiology is unclear. Previous studies have revealed several potential causes of this disease, such as genetic abnormalities, dysregulation of the immune system, inflammation, and environmental factors. Recently, interactions between the gut and the brain in ASD have received considerable attention in scientific and medical research. ASD has been shown to have a significant gut microbiome component, as recent studies found that autistic individuals harbor an altered gut bacterial microbiota. Over many years, selected microbiota have become resident in the human gastrointestinal tract, which is integrated with the immune system, metabolism and nervous system. The indigenous microbiota of the colon also provides an important host defense by inhibiting the growth of potentially pathogenic microorganisms, such as Clostridium species. Gastrointestinal problems, including constipation, abdominal pain, gaseousness, diarrhea, and flatulence, are common symptoms associated with ASD. In some cases, remodeling the gut microbiota by therapeutic interventions, such as antibiotic administration and microbiota transfer therapy have reported alleviated the symptoms of ASD.

Biomarker screening, including screening through metabolite analysis, which can be performed any time after birth, represents an attractive addition to the ASD screening toolkit. Although measurements of concentrations of bacterial metabolites in fecal or stool samples could provide useful to indicate gut bacterial species abundance, the collection and processing of fecal or stool specimens can be challenging. In health care settings, other types of biological samples (e.g., blood, urine) are much more frequently used for diagnostic testing due to ease of collection and handling and the ability to collect specimens in a timely manner.

Several biochemical metabolic markers have been associated with autistic traits. For example, the presence of metabolic abnormalities associated with ASD may include phenylketonuria (“PKU”), disorders in purine metabolism, folate deficiency in brain development, succinic semialdehyde dehydrogenase deficiency, Smith-Lemli-Optiz syndrome (“SLOS”), organic acidurias (e.g., pyridoxine dependency, 3-methylcrotonyl-CoA carboxylase deficiency, and propionic acidemia), and mitochondrial disorders. These metabolic abnormalities may originate in part from an altered gut microbiome from ASD individuals where there is the presence of pathogenic microorganisms. While these isolated findings have provided additional insight into the biochemical pathways that may be involved in ASD, a reliable testing protocol for determining the presence or susceptibility of ASD with demonstrated broad-based success in ASD diagnosis and treatment, particularly among young children, has not been described. Furthermore, there are hundreds of other metabolites that have not been identified and linked to clinical outcomes of ASD. In sum, no currently reported biomarker holds promise as early developmental screen or an early diagnostic or prognostic tool for pediatric settings at young ages from birth through early childhood when these clinical tools are most needed.

Accordingly, an improved method is needed that can offer a reliable confirmation of the clinical diagnosis of behavioural traits characteristics of the presence or predisposition of developing ASD and treatment thereof. The need also exists for objective screening and treatment of individuals with ASD without solely relying on behavioural features because of the absence of consistent physical characteristics in ASD and uncertainty of the timing of the manifestations of those characteristics in the first few years of life. The need also exists for a test that permits the earlier diagnosis of ASD in subjects, particularly in early childhood (i.e., children age 3 years or younger) and early treatment thereof.

### SUMMARY

As embodied and described herein, in one aspect, the present disclosure relates to a method for diagnosing and treating Autism Spectrum Disorder (“ASD”) in a subject. The method comprises: (a) providing a biological sample obtained from the subject; (b) measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (“HPHPA”), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample; (c) comparing the concentration levels of the ASD-related metabolites from the obtained sample to the concentration levels of reference ASD-related metabolites from an ASD-negative sample; (d) identifying the subject as having ASD if the concentration levels of the ASD-related metabolites from the obtained sample is different relative to the concentration levels of the reference ASD-related metabolites from the ASD-negative sample; and (e) treating the subject so identified with an ASD treatment regime.

As embodied and described herein, in another aspect, the present disclosure also relates to a method for diagnosing and treating ASD in a subject. The method comprising: (a) providing a biological sample obtained from the subject; (b) measuring or having measured in a spectroscopy unit the concentration levels of at least one, at least two, at least three, at least four or at least five ASD-related metabolite selected from the group consisting of fumaric acid, L-malic acid, 4-hydroxymandelic acid, 2-hydroxyisovaleric acid, 3-(3-Hydroxyphenyl)-3-hydroxypropanoic acid HPHPA, p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample; (c) comparing or having compared concentration levels of the ASD-related metabolites as determined in the spectroscopy unit to the concentration levels of reference ASD-related metabolites from an ASD-negative sample; (d) identifying the subject as having ASD if the concentration levels of the ASD-related metabolites from the obtained sample are different relative to the concentration levels of the reference ASD-related metabolites from the ASD-negative sample; and (e) treating the subject so identified with an ASD treatment regime.

As embodied and described herein, in yet another aspect, the present disclosure also relates to a method of monitoring ASD progression and treating the ASD in a subject. The method comprising: (a) providing a first biological sample obtained from the subject at a first time; (b) assessing a first ASD-related metabolite profile by measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the first obtained sample; (c) comparing the first ASD-related metabolite profile with a reference ASD-related metabolite profile from an ASD-negative sample; (d) determining that there is a first difference between the first ASD-related metabolite profile and the reference ASD-related metabolite profile from the ASD-negative sample, the first difference being indicative of ASD; (e) providing a second biological sample obtained from the subject at a second time that is after the first time; (f) assessing a second ASD-related metabolite profile by measuring concentration level of the ASD-related metabolites from the second obtained sample; (g) comparing the second ASD-related metabolite profile with the reference ASD-related metabolite profile from the ASD-negative sample; (h) determining that there is a second difference between the first ASD-related metabolite profile and the reference ASD-related metabolite profile from the ASD-negative sample, the second difference being indicative of ASD; (i) determining ASD progression based on at least in part on the first and second differences; and (j) treating the subject as identified with an ASD treatment regime.

As embodied and described herein, in yet another aspect, the present disclosure also relates to a kit for use in any one of the methods as described herein, comprising reagents for measuring the concentration levels of the ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose, optionally together with instructions for use.

As embodied and described herein, in yet another aspect, the present disclosure also relates to a metabolomic profile for Autism Spectrum Disorder (“ASD”). In some aspects, the metabolic profile for ASD comprises one or more, preferably at least two, at least three, at least four or at least five of:
(a) fumaric acid and/or L-malic acid at decreased concentration levels of reference fumaric acid and/or reference L-malic acid from an ASD-negative sample;
(b) 3-(3-Hydroxyphenyl)-3-hydroxypropanoic acid (HPHPA) at increased concentration level of reference 3-(3-Hydroxyphenyl)-3-hydroxypropanoic acid (HPHPA) from an ASD-negative sample;
(c) acylcarnitine, preferably selected from C10:1, C16:2 and/or C7-DC, at increased concentration levels of reference acylcarnitine, preferably selected from C10:1, C16:2 and/or C7-DC, from an ASD-negative sample;
(d) lysophospholipid, preferably lysoPC a C17:0, and/or lysoPC a C20:3, at increased concentration levels of reference lysophospholipid, preferably lysoPC a C17:0, and/or lysoPC a C20:3, from an ASD-negative sample;
(e) sphingolipid, preferably SM (OH) C24:1 and/or SM (OH) C22:2, at increased concentration levels of reference sphingolipid, preferably SM (OH) C24:1 and/or SM (OH) C22:2, from an ASD-negative sample;
(f) glycerophospholipid, preferably PC ae C36:0 and/or PC aa C40, at increased concentration levels of reference glycerophospholipid, preferably PC ae C36:0 and/or PC aa C40, from an ASD-negative sample; and
(g) 4-hydroxymandelic acid and/or the 2-hydroxyisovaleric acid at decreased concentration levels of reference 4-hydroxymandelic acid and/or reference 2-hydroxyisovaleric acid from an ASD-negative sample.

In other aspects, the metabolomic profile comprises: (a) fumaric acid at a concentration level of at least about 2 times or less than the median concentration levels of reference fumaric acid from non-ASD subjects; and (b) L-malic acid at a concentration level of at least about 2 times or less than the median concentration levels of reference L-malic acid from non-ASD subjects. Preferably, the foregoing metabolomic profile further comprising: (c) HPHPA at a concentration level of at least about 10 times or greater than the median concentration level of reference HPHPA from non-ASD subjects.

As embodied and described herein, in yet another aspect, the present disclosure also relates to a proteomic profile for Autism Spectrum Disorder (“ASD”). In some aspects, the proteomic profile for ASD comprises one or more, preferably at least two, at least three, at least four or at least five of:
(a) alpha-2-antiplasmin, coagulation factor X, coagulation factor XI and/or tenascin C at increased concentration levels of reference alpha-2-antiplasmin, reference coagulation factor X, reference coagulation factor XI and/or reference tenascin C from an ASD-negative sample; and
(b) coagulation factor XIII A chain, thrombospondin-1 (TSP-1) and/or retinol-binding protein 4 (RBP4) at decreased concentration levels of reference coagulation factor XIII A chain, thrombospondin-1 (TSP-1) and/or retinol-binding protein 4 (RBP4) from an ASD-negative sample.

As embodied and described herein, in yet another aspect, the present disclosure also relates to a kit for diagnosis of ASD. The kit comprises: (a) a detector configured to detect concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxy phenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from an obtained biological sample; (b) a composition comprising fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose in a control levels corresponding to a control group of ASD-negative people; (c) a multivariate analysis system configured to analyze a difference in the concentration levels of the ASD-related metabolites and the control levels; and (d) optionally, instruction for an ASD diagnosis method; wherein the method comprises measuring, using the detector, the levels of the ASD-related metabolites from the obtained biological sample, and comparing the levels of the obtained ASD-related metabolites to the control levels of the ASD-related metabolites.

As embodied and described herein, in yet another aspect, the present disclosure also relates to a computer-implemented method for processing biological sample of a subject, diagnosing an Autism Spectrum Disorder (ASD) and treating the ASD. The computer-implemented method comprises: (a) receiving a biological sample obtained from the subject; (b) processing the sample in a spectroscopy unit directly or wirelessly linked to a processing device, the processing device having memory for storing measurement data from the spectroscopy unit; (c) in the spectroscopy unit, measuring levels of least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose and storing the measurement data in the processor; (d) comparing the stored measurement data to a value in the memory representing an ASD-negative sample using multivariate statistical analysis; (e) storing on the processing device a result corresponding to at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucoses from the obtained sample, wherein the result identifies the subject as having ASD if the measurement data representing the level of the ASD-related metabolites are different relative to a concentration levels of reference ASD-related metabolites from an ASD-negative sample; and (f) displaying an ASD treatment regime on an electronic display connected directly or wirelessly to the processor for the subject identified as having ASD or as having predisposition of developing ASD, the displayed treatment regime comprising electronic text on a graphical user interface describing one or more of: (i) dietary adjustments; (ii) nutritional supplements; (iii) behaviour training or a combination thereof, to the subject diagnosed as having or predisposed of developing the ASD; and/or (iv) adjusting the blood levels of one or more of the ASD-related metabolites in the subject diagnosed as having ASD or predisposed of developing the ASD until an improvement in the behavioral performance in the subject is observed.

As embodied and broadly described herein, in yet another aspect, the present disclosure also relates to a method for diagnosing and treating Autism Spectrum Disorder (ASD) in a subject. The method comprises: (a) providing a biological sample obtained from the subject; (b) comparing the concentration levels of the ASD-related metabolites from the obtained sample to the concentration levels of reference ASD-related metabolites from an ASD-negative sample; (c) measuring concentration levels of at least one, at least two, at least three, at least four or at least five ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C; (d) identifying the subject as having ASD if the concentration levels of the ASD-related proteins from the obtained sample are different relative to the concentration levels of the reference ASD-related proteins from the ASD-negative sample; and (e) treating the subject so identified with an ASD treatment regime.

As embodied and broadly described herein, in yet another aspect, the present disclosure also relates to a method for diagnosing and treating Autism Spectrum Disorder (ASD) in a subject. The method comprises: (a) providing a biological sample obtained from the subject (b) measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample; (c) comparing the concentration levels of the ASD-related metabolites from the obtained sample to the concentration levels of reference ASD-related metabolites from an ASD-negative sample; (d) measuring concentration levels of at least one, at least two, at least three, at least four or at least five ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C; (e) comparing the concentration levels of the ASD-related proteins from the obtained sample to the concentration levels of reference ASD-related proteins from an ASD-negative sample; (0 identifying the subject as having ASD if the concentration levels of the ASD-related metabolites from the obtained sample are different relative to the concentration levels of the reference ASD-related metabolites from the ASD-negative sample, and the concentration levels of the ASD-related proteins from the obtained sample are different relative to the concentration levels of the reference ASD-related proteins from the ASD-negative sample; and (g) treating the subject so identified with an ASD treatment regime.

As embodied and broadly described herein, in yet another aspect, the present disclosure also relates to a kit for use in any one of the methods as described herein, comprising reagents for measuring the concentration levels of the ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C, optionally together with instructions for use.

As embodied and described herein, in yet a further aspect, the present disclosure also relates to a computer-implemented method to diagnose a subject as having ASD or predisposed to developing same, or to assess progression/regression of ASD, the method comprising: receiving and inputting a data set into a memory of a computer, the data set comprising a metabolic and proteomic profile associated with the subject; identifying whether the metabolic and proteomic profile is indicative of ASD, predisposition to developing ASD, or progression or regression thereof based on ASD modelling data from a previous computer-implemented modelling analysis; and displaying an ASD treatment regime on an electronic display connected directly or wirelessly to a processor of the computer for the subject, the displayed treatment regime comprising electronic text on a graphical user interface displaying at least one of: (i) one or more dietary adjustments; (ii) one or more nutritional supplements; (iii) behaviour training or a combination thereof, to the subject diagnosed as having or predisposed of developing the ASD; and/or (iv) adjusting the blood levels of one or more of the ASD-related metabolites in the subject diagnosed as having ASD or predisposed of developing the ASD until an improvement in the behavioral performance in the subject is observed.

Examples of biomarkers in the metabolic and proteomic profile including those described above and further herein. In one embodiment, between 1 and 50, 2 and 40 or 5 and 30 biomarkers are assessed as based on the ASD modelling data.

As embodied and described herein, in yet a further aspect, the present disclosure also relates to a computer-implemented method to diagnose a subject as having ASD or predisposed to developing same, or to assess progression/regression of ASD, based on analyzing proteomic and/or metabolic profiles of the subject, preferably both profiles. Such method comprises: receiving and inputting a data set in a memory of a computer, the data set comprising measurements of a plurality of ASD proteomic and/or metabolic biomarkers obtained from a biological sample from the subject. In one embodiment, the dataset comprises at least one of a metabolic and proteomic profile associated with the subject, and/or comprises at least 2, 3, 4, 5, 6, 8, 9 or 10 biomarkers that are pre-determined from metabolic and/or proteomic ASD computer modelling as being indicative of ASD diagnosis and/or progression of ASD. One embodiment comprises previously obtaining computer modelling data from test and control groups in computer-readable format based on previously obtained computer-implemented calculations that, in some embodiments, assign a weight to a given biomarker or set of markers within the at least one metabolic and/or proteomic profiles obtained from ADS and control subjects, preferably both profiles. The method may further comprise performing, with the computer, data calculations on the inputted data set, the calculations based on comparing biomarker data obtained from the previous computer modelling data with the inputted data set, and identifying the subject as having ASD, having a predisposition of developing ASD or assessing progression/regression of ASD based on results from said computer-implemented comparison.

In some embodiments, the method may further comprise, based on the comparison, displaying an ASD treatment regime on an electronic display connected directly or wirelessly to a processor of the computer for the subject, the displayed treatment regime comprising electronic text on a graphical user interface displaying at least one of: (i) one or more dietary adjustments; (ii) one or more nutritional supplements; (iii) behaviour training or a combination thereof, to the subject diagnosed as having or predisposed of developing the ASD; and/or (iv) adjusting the blood levels of one or more of the ASD-related metabolites in the subject diagnosed as having ASD or predisposed of developing the ASD until an improvement in the behavioral performance in the subject is observed.

In one embodiment of any one of the foregoing aspects of the disclosure, the data set is spectroscopy data obtained from a spectroscopy unit. In another embodiment, the data set is obtained from a high-throughput measurement unit.

As mentioned, in one embodiment, the previous modelling data identifies a plurality of biomarkers from proteomic and/or metabolic profiles of subjects with and without ASD.

In one embodiment, the modelling data is obtained from ASD test and control group data, each group data subjected to a computer-implemented calculation comprising at least one of: a computer-generated Receiver Operating Characteristic (ROC) curve analysis, a Principal Component Analysis (PCA) plot, and a latent structures discriminant analysis (PLS-DA) model and/or a Variable Importance of Projection (VIP) plot and thereby obtaining a set of at least 2, 3, 4, 5, 6, 7, 8, 9 or 10 biomarkers identified as contributing to the diagnosis, development or regression of ASD relative to other biomarkers measured.

In some non-limiting examples, at least 2, 3, 4, or 5 metabolic and/or proteomic biomarkers are measured. In further embodiments, up to 100, 95, 90, 85, 80, 75, 70, 65, 60, 55, 50, 45, 40, 35, 30, 25 or 20 metabolic and/or proteomic biomarkers are identified based on the computer modelling. Preferably, the biomarkers are selected from both proteomic and metabolic profiles obtained from computer modelling.

In some embodiments, the biomarkers are selected from computer modelling based on assigning a weight to biomarkers selected from a proteomic and metabolic profile, the weight based on the ability of the marker to diagnose or assess ASD progression.

In one embodiment, the biomarkers are selected from at least one of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample from the subject.

All features of exemplary embodiments which are described in this disclosure and are not mutually exclusive can be combined with one another. Elements of one embodiment can be utilized in the other embodiments without further mention. Other aspects and features of the present disclosure will become apparent to those ordinarily skilled in the art upon review of the following description of specific embodiments in conjunction with the accompanying Figures.

### BRIEF DESCRIPTION OF THE DRAWINGS

While the specification concludes with claims particularly pointing out and distinctly claiming the disclosure, it is believed that the disclosure will be better understood from the following description of the accompanying figures wherein:

FIG. 1 includes a table of the urine organic acids tested.

FIG. 2 is a flowchart of the GC-/LC-MS and analysis process.

FIG. 3 is a visualization of the PCA and PLS-DA plots of the metabolites tested from the urine samples (Example 1).

FIG. 4 is a graph of the ROC curve of the metabolites tested from Example 1.

FIG. 5 is a Variable Importance of Projection (VIP) plot of the metabolites tested from Example 1.

FIG. 6 is a visualization of the PCA and PLS-DA plots of the metabolites tested from the serum samples (Example 2).

FIG. 7 is a Variable Importance of Projection (VIP) plot of the metabolites tested from Example 2.

FIG. 8 is a visualization of the PCA and PLS-DA plots of the proteomics tested from the plasma samples (Example 2).

FIG. 9 is a Variable Importance of Projection (VIP) plot of the metabolites tested from Example 2.

FIG. 10A is a graph of the ROC curve of the metabolites tested from Example 2.

FIG. 10B is a graph of the ROC curve of the proteomics tested from Example 2.

In the drawings, exemplary embodiments are illustrated by way of example. It is to be expressly understood that the description and drawings are only for the purpose of illustrating certain embodiments and are an aid for understanding. They are not intended to be construed as limiting to the invention in any manner.

### DETAILED DESCRIPTION

A detailed description of one or more embodiments of the invention is provided below along with accompanying figures that illustrate the principles of the invention. The invention is described in connection with such embodiments, but the invention is not limited to any particular embodiment described herein. The scope of the invention is limited only by the claims and equivalents thereof. Numerous specific details are set forth in the following description in order to provide a thorough understanding of the invention. These details are provided for the purpose of providing non-limiting examples and the invention may be practiced according to the claims without some or all of these specific details. For the purpose of clarity, certain technical material that is known in the technical fields related to the invention has not been described in detail so that the invention is not unnecessarily obscured by such descriptions.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

Articles such as “a” and “an” when used in a claim, are understood to mean one or more of what is claimed or described.

The terms “autism” and “autism spectrum disorder” (“ASD”) are used interchangeably to generally describe ASD as identified by a qualified individual using the standard behavioral testing protocols/guidelines as described in the Infant Toddler Checklist (“ITC”, and also known as the Communication and Symbolic Behaviour Scales and Development Profile) and the Modified Checklist for Autism in Toddlers (“M-CHAT”). The term “ASD” may also refer to a pathological condition with one or more of the symptoms of ASD including but not limited to anxiety, Fragile X, Rett syndrome, tuberous sclerosis, obsessive compulsive disorder, attention deficit disorder, schizophrenia, autistic disorder (classic autism), Asperger's disorder (Asperger syndrome), pervasive developmental disorder not otherwise specified (“PDD-NOS”), or childhood disintegrative disorder (“CDD”).

The term “ASD-negative” generally refers to a biological sample from an individual that does not suffer from ASD or developing ASD.

The term “ASD treatment regime” generally refers to an intervention made in response to a subject suffering from ASD. The aim of the regime may include, but is not limited to, one or more of the alleviation or prevention of symptoms, slowing or stopping the progression or worsening of ASD and the remission of ASD. In some embodiments, “ASD treatment regime” refers to therapeutic treatment (e.g., changing ASD-related metabolite levels), dietary adjustments, nutritional supplements and/or behavioral training.

The terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

The term “improvement in behavioral performance” generally refers to prevention or reduction in the severity or frequency, to whatever extent, of one or more of the behavioral disorders, symptoms and/or abnormalities expressed by individual suffering from ASD, or a pathological condition with one or more of the symptoms of ASD. Non-limiting examples of the behavioral symptoms include repetitive behavior, decreased pre-pulse inhibition (“PPI”), and increased anxiety. The improvement may be observed by the individual undertaking the treatment or by another person (i.e., medical or otherwise).

The term “metabolite” generally refers to any molecule involved in metabolism. Metabolites can be products, substrates or intermediates in metabolic processes. Metabolites may include, without limitation, amino acids, peptides, acylcarnitines, monosaccharides, lipids and phospholipids, lysophospholipid, sphingolipids, glycerophospholipids, glucose, prostaglandins, hydroxyeicosatetraenoic acids, hydroxyoctadecadienoic acids, steroids, bile acids, and glycolipids and phospholipids.

The term “ASD-related metabolite” or “metabolomic profile” generally refers to a profile of metabolites associated with ASD comprising one, or two or more metabolites selected from fumaric acid, L-malic acid, 4-hydroxymandelic acid, 2-hydroxyisovaleric acid, 3-(3-Hydroxyphenyl)-3-hydroxypropanoic acid (“HPHPA”), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose or a combination thereof.

The terms “preferred”, “preferably” and variants generally refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The term “preventing” and “prevention” are used interchangeably and generally refer to any activity that leads to a reduction in risk of developing ASD in the subject.

The term “ASD-related protein” or “proteomic profile” generally refers to a profile of proteins associated with ASD comprising two or more, three or more, four or more, or five or more proteins selected from retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C or a combination thereof.

The term “subject” generally refers to a vertebrate, such as a mammal. The term “mammal” is defined as individual belonging to the class Mammalia and includes, without limitation, humans, domestic and farm animals, and zoo, sports or pet animals, such as sheep, dogs, horses, cats or cows. In some embodiments, the subject is human.

The term “treating” or “treatment” generally refers to an intervention made in response to ASD or associated symptoms manifested by a subject. The aim of treatment may include, but is not limited to, one or more of the alleviation or prevention of ASD, slowing or stopping the progression or worsening of ASD and the remission of ASD. In certain embodiments, “treatment” refers to therapeutic, dietary, supplemental and/or behavior therapy.

In all embodiments of the present disclosure, all percentages, concentrations, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (° C.), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

### Method of Diagnosing and Treating ASD

In one broad aspect, the present disclosure relates to methods for (early) diagnosis and treatment of ASD, and any associated symptoms, in a subject. The disclosure is predicated, at least in part, on the identification of new metabolites and/or new proteins that provide etiological information related to ASD and provides an opportunity for objective metabolite-based and/or protein-based diagnosis of a subject's ASD that can lead to more effective therapy. Given the complexities of the interactions between genetics and the environment, metabolic and/or proteomic profiling can provide an important approach towards a better understanding of ASD and the development of diagnostic tests that aid in individualized treatment decisions. Metabolism and/or proteomic based analysis has the advantage to identify biomarker profiles derived from an individual's inherited genes as well as capture the interactions of the individual's current lifestyle behaviors (e.g., smoking, alcohol consumption, sleep behaviours, physical activity and the like), gut microbiome, dietary, and environmental factors that contribute to the unique metabolic profile and/or protein profile of a subject with ASD. Combining early diagnoses and an ASD treatment regime has the further advantage of increased positive therapeutic outcomes earlier on in the subject's life. Described herein are methods that provide for the identification of new metabolic profiles and/or proteomic profiles among subjects with ASD that serve to diagnose and treat those subjects. Therefore, the present disclosure provides an advancement in the art.

With the present disclosure, a new metabolomic profile for ASD is identified in the subject having ASD. The metabolomic profile for ASD comprises at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose. Preferably, the metabolomic profile associated with ASD comprises fumaric acid, L-malic acid and 3-(3-Hydroxyphenyl)-3-hydroxypropanoic acid (HPHPA).

The metabolite 3-(3-Hydroxyphenyl)-3-hydroxypropanoic acid (“HPHPA”) is identified as a biomarker of ASD and observed to be elevated to a level of about 10 times or greater compared to a median level of a reference HPHPA in ASD-negative individuals. In some aspects, an elevated HPHPA level of about 100 μmol/mmol creatinine or more identifies the subject as having ASD. Such a subject may have been clinically diagnosed with ASD and/or is undergoing treatment for ASD. HPHPA is an abundant organic acid detected in human urine and is thought to be from nutritional sources. However, it has been recently reported that HPHPA in urine may arise from an abnormal phenylalanine metabolite arising from bacterial (i.e., Clostridia species) metabolism of polyphenols in the gastrointestinal tract.

The metabolite fumaric acid is also identified as a biomarker of ASD and observed to be depressed to a level of about 2 times or less compared to a median level of a reference fumaric acid in ASD-negative individuals. In some aspects, a decreased fumaric acid level of about 0.4 μmol/mmol creatinine or more identifies the subject as having ASD. Such a subject may have been clinically diagnosed with ASD and/or is undergoing treatment for ASD. Fumaric acid is created through the metabolism in the Krebs tricarboxylic acid (TCA) cycle where fumaric acid is a precursor to L-malic acid. It can be excreted in urine and low levels of fumaric acid may indicate mitochondrial dysfunction.

The metabolite L-malic acid is also identified as a biomarker of ASD and observed to be depressed to a level of about 2 times or lesser compared to a median level of a reference L-malic acid in ASD-negative individuals. In some aspects, a decreased fumaric acid level of about 7 μmol/mmol creatinine or more identifies the subject as having ASD. Such a subject may have been clinically diagnosed with ASD and/or is undergoing treatment for ASD. L-malic acid, like fumaric acid, is an intermediate in the Krebs TCA cycle and created through the metabolism of citric acid. L-malic acid is a precursor to oxaloacetic acid. It can be excreted in urine and low levels of L-malic acid may indicate mitochondrial dysfunction.

The metabolite acylcarnitine is also identified as a biomarker of ASD and observed to be increased to a level of from about 0.5 to about 2.0, preferably from about 0.75 to about 1.75, or preferably from about 0.85 to about 1.5 times or greater compared to a median level of a reference acylcarnitine in ASD-negative subjects. Preferably, the acylcarnitine is selected from C10:1 (decenoylcarnitine), C16:2 (9,12-hexadecadienoylcarnitine) and/or C7-DC (pimelyl-L-carnitine). Such a subject may have been clinically diagnosed with ASD and/or is undergoing treatment for ASD. Mitochondrial dysfunction as a cause of ASD has been postulated previously based on biochemical, genetic and histopathological findings. Additionally, symptoms of a subset of children with ASD closely overlapped with criteria for mitochondrial respiratory chain disorders in a population-based survey (Oliveira, G. et al., Mitochondrial dysfunction in autism spectrum disorders: a population-based study. Dev. Med. Child Neurol. 47, 185-189 (2005)). As the mitochondria is a central organelle responsible for providing cells with energy to function, defects in its function can result in a wide range of health concerns, including fatigue, weakness, metabolic stroke, developmental or cognitive disabilities, and impairment of gastrointestinal or kidney function, some of which are observed as ASD symptoms. Evaluation of mitochondrial function in plasma and increased levels of acylcarnitine may indicate mitochondrial dysfunction. Carnitine is involved in fatty acid (FA) metabolism, playing an obligate role in the mitochondrial oxidation of long-chain FA and buffering intracellular acyl-CoA-CoA ration. As such, acylcarnitine profiles suggest mitochondrial dysfunction through direct or indirect disruption of β-oxidation. In the context of ASD, acylcarnitines are found to be dysregulated. Without wishing to be bound by theory, plasma acylcarnitines can be elevated due to either extensive inhibition of acylcarnitine uptake or decreased intramitochondrial substrate availability, possibly of CoA to sustain fatty acyl-CoA formation inside the mitochondria.

The metabolite lysophospholipid is also identified as a biomarker of ASD and observed to be increased to a level of about 1.1 or greater, or preferably about 1.2 times or greater compared to a median level of a reference lysophospholipid in ASD-negative subjects. Preferably the lysophospholipid is lysoPC a C17:0, and/or lysoPC a C20:3. Elevated levels of lysophospholipids are found in serum of ASD subjects. Such a subject may have been clinically diagnosed with ASD and/or is undergoing treatment for ASD. Without wishing to be bound by theory, elevated levels of serum lysophospholipids are indicative of disturbance in fatty acid metabolism.

The metabolite sphingolipid is also identified as a biomarker of ASD and observed to be increased to a level of about 1.05, or preferably about 1.1 times or greater compared to a median level of a reference sphingolipid in ASD-negative individuals. Preferably, the sphingolipid is SM (OH) C24:1 (C24:1 hydroxysphingomyelin) and/or SM (OH) C22:2 (C22:2 hydroxysphinogomyelin). Elevated levels of sphingolipids are found in serum of ASD subjects. Such a subject may have been clinically diagnosed with ASD and/or is undergoing treatment for ASD. Without wishing to be bound by theory, elevated levels of serum sphingolipids are indicative of disturbance in fatty acid metabolism.

The metabolite glycerophospholipid is also identified as a biomarker of ASD and observed to be increased to a level of about 1.05, or preferably about 1.1 times or greater compared to a median level of a reference glycerophospholipid in ASD-negative individuals. Preferably, the glycerophospholipid is PC ae C36:0 (Phosphatidylcholine with acyl-alkyl residue sum C36:0) and/or PC aa C40:2 (Phosphatidylcholine with diacyl residue sum C40:2). Elevated levels of glycerophospholipids are found in serum of ASD subjects. Such a subject may have been clinically diagnosed with ASD and/or is undergoing treatment for ASD. Without wishing to be bound by theory, elevated levels of serum glycerophospholipids are indicative of disturbance in fatty acid metabolism.

The metabolite glucose is also identified as a biomarker of ASD and observed to be increased to a level of about 1.2 or preferably about 1.2 times or greater compared to a median level of a reference glucose in ASD-negative individuals. Elevated level of glucose is found in serum of ASD subjects. Such a subject may have been clinically diagnosed with ASD and/or is undergoing treatment for ASD. In addition to fatty acid oxidation, glycolysis is another major process for energy production. Recent study has elucidated the connection between abnormal neonatal glucose level and mitochondrial dysfunction (Hoirisch-Clapauch, S. & Nardi, A. E. Autism spectrum disorders: let's talk about glucose? Transl. Psychiatry 9, 51-51 (2019)). There seems to be a link between glucose metabolism, mitochondrial dysfunction and neurological symptoms of ASD. Moreover, low levels of insulin-like growth factors, as seen in the ASD group, have been observed in neurological diseases in children.

The metabolites 4-hydroxymandelic acid and/or the 2-hydroxyisovaleric acid are also identified as biomarkers of ASD and observed to be absent (or found in lower than quantifiable levels (i.e., undetectable)) in ASD samples when compared to a median level of a reference 4-hydroxymandelic acid and/or the 2-hydroxyisovaleric acid in ASD-negative individuals. Decreased or undetected levels of 4-hydroxymandelic acid and/or the 2-hydroxyisovaleric acid are found in urine of ASD subjects. Such a subject may have been clinically diagnosed with ASD and/or is undergoing treatment for ASD. These organic acids are lower than quantifiable levels in samples from ASD subjects as compared to non-ASD subjects.

With the present disclosure, a new proteomic profile for ASD is identified in the subject having ASD. The proteomic profile for ASD comprises at least one, at least two, at least three, at least four or at least five ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C.

The inventors identified elevated levels of proteins involved in blood clotting in ASD subjects. In particular, the proteins alpha-2-antiplasmin, coagulation factor X, and coagulation factor XI are identified as biomarkers of ASD and observed to be increased to a level of from about 0.5 to about 1.5, preferably from about 0.75 to about 1.3, or preferably from about 0.85 to about 1.2 times or greater compared to a median level of reference alpha-2-antiplasmin, coagulation factor X, and coagulation factor XI in ASD-negative subjects. These proteins support the process of blood coagulation, and when activated dissolve fibrin.

The protein coagulation factor XIII A chain is also identified as a biomarker of ASD and observed to be decreased to a level of about 1.2 times or less, or preferably about 1.3 times or lesser compared to a median level of a reference coagulation factor XIII A chain in ASD-negative subjects. This protein also supports the process of blood coagulation and dissolves fibrin when activated. Decreased levels of coagulation factor XIII A chain indicate dysregulation of blood clotting process taking place in ASD subjects. Without wishing to be bound by theory, fibrin is involved in the final stage of the coagulation cascade, which requires coagulation factor XIII A chain to stabilize the network of fibrin. Decreased levels of coagulation factor XIII A chain disrupt the normal blood clotting process.

The protein thrombospondin-1 (TSP-1) is also identified as a biomarker of ASD and observed to be decreased to a level of about 1.2 time or less, preferably about 1.4 time or less, or preferably about 1.5 times or less compared to a median level of a reference thrombospondin-1 (TSP-1) in ASD-negative subjects. Thrombospondin-1 (TSP-1) is a substrate of coagulation factor XIII during the activation of platelets and decreased levels of Thrombospondin-1 (TSP-1) also disrupt the normal blood clotting process.

The protein tenascin C is also identified as a biomarker of ASD and observed to be increased to a level of about 0.3 times or greater, preferably about 0.35 times or greater, or preferably 0.4 times or greater compared to a median level of a reference tenascin C in ASD-negative subjects. Tenascin C functions as a trigger for innate immunity and immunoregulation. Elevated levels of tenascin C increase proinflammatory cytokines seen during injury or infection, a transient increase during the progression of inflammation and wound healing, and persistent expression associated with inflammatory, autoimmune and fibrotic disease.

The protein retinol binding protein 4 (RBP4) is also identified as a biomarker of ASD and observed to a level of about 1.5 times or less, preferably about 1.7 times or less or preferably about 1.8 times or less compared to a median level of a reference retinol binding protein 4 (RBP4) in ASD-negative subjects. RBP4 levels are a surrogate marker for retinol status due to its function in binding and transporting vitamin A metabolite retinol. There is no known disclosure of the association of plasma RBP4 and autism. The decreased levels of RBP4 may be attributable to the acute phase response (inflammatory states) and malnutrition. Without wishing to be bound by theory, taken together, an acute phase response, reduced functions in coagulation and dysregulations of endothelial and perivascular cells may be forming the basis of the ASD phenotype, particularly in developmental delay and changes in the immune system.

It has been surprisingly discovered that the metabolomic profile and/or proteomic profile are altered in a subject suffering from ASD as compared to non-autistic individual and/or an ASD-negative individual. In particular, the levels of the ASD-related metabolites and/or ASD-related proteins are altered in circulation of the subject having ASD as compared to a non-autistic individual. In certain embodiments, the levels of the ASD-related metabolites and/or ASD-related proteins are altered in the blood (e.g., serum, plasma), body fluids (e.g., cerebrospinal fluid, pleural fluid, amniotic fluid, semen, or saliva), urine, and/or feces of the subject having ASD. Without wishing to be bound by theory, it is believed that the ASD-related metabolites and/or ASD-related proteins play a causative role in the development of ASD-related behaviors in the subject having ASD. Alternative, the alteration in the level of the ASD-related metabolites and/or ASD-related proteins are caused by the ASD.

Specifically, in one aspect, the present disclosure provides for a method for diagnosing and treating Autism Spectrum Disorder (“ASD”) in a subject. The method comprises step (a) providing a biological sample obtained from the subject, preferably a human. The biological sample may be from an adult subject or a teenager. The biological sample may also be obtained from a child, for example, a child that is under about 10 years of age, under about 5 years of age, under about 3 years of age, under about 2 years of age, or under about 18 months of age. In accordance with the methods disclosed herein, any type of biological sample that originates anywhere from the body of a subject may be tested, including but not limited to, blood (including, but not limited to serum or plasma), cerebrospinal fluid (“CSF”), pleural fluid, urine, stool, sweat, tears, breath condensate, saliva vitreous humour, a tissue sample, amniotic fluid, a chorionic villus sampling, brain tissue, a biopsy of any solid tissue including tumor, adjacent normal, smooth and skeletal muscle, adipose tissue, liver, skin, hair, brain, kidney, pancreas, lung or the like may be used. Preferably, the biological sample obtained from a live subject is urine. The ASD-related metabolites may be extracted from their biological source using any number of extraction/clean-up procedures that are typically used in quantitative analytical chemistry.

The method further comprises step (b) measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample. In certain embodiments, the method comprises measuring at least 6, 7, 8, 9, 10, 11, 12, 13, 14 or 15 ASD-related metabolites from the obtained sample.

In certain embodiments, the measurement of the concentration levels of the ASD-related metabolites may be through mass spectrometry, including but not limited to gas chromatography mass spectrometry (GC-MS) GC and liquid chromatography mass spectrometry (e.g., LC-MS, LC-MS-MS, LC-MRM, LC-SIM, and LC-SRM). Preferably, the ASD-related metabolites are measured by a spectroscopic technique, wherein the spectroscopic technique is selected from the group consisting of liquid chromatography, gas chromatography, liquid chromatography mass spectrometry, gas chromatography mass spectrometry, high performance liquid chromatography mass spectrometry, capillary electrophoresis mass spectrometry, nuclear magnetic resonance spectrometry (NMR), raman spectroscopy, and infrared spectroscopy. The measurement may also be performed under other methodology, such as for example, a colorimetric, enzymatic, immunological methodology, and gene expression analysis including, for example, real-time PCR, RT-PCT, northern analysis, and in situ hybridization.

In certain embodiments, with any of the methods described herein, the methods may further include measuring the concentration levels of one or more additional ASD-related metabolites, including, but not limited to, any of those described herein may also be measured. The novel approach of the present disclosure identifies biomarkers that have high predictive value for a subset of the diagnostic class (i.e., ASD in this case). The advantage of including additional ASD-related metabolites is to give rise to the opportunity to reveal additional metabolite sub-types and increase the overall sensitivity of the method. Non-limiting examples of additional ASD-relates metabolites are provided in Table 1. Wherein the subject is identified as having ASD if the concentration level of the one or more additional ASD-related metabolites obtained from the biological sample is different to that in a reference ASD-negative sample.

TABLE 1
Exemplary Additional ASD-related Metabolites

| | 3-hydroxy-3- | |
|---|---|---|
| | methylbutyric | Pregnenolone |
| N-acetylserine | Acid ^{l} | sulfate ^{l} |
| Imidazole | 3-methyl-2-oxovaleric | Lyso PE (22:6) ^{l} |
| | acid ^{l} | |
| Propionate | Salicylic acid ^{l} | Glycine ^{l} |
| Serotonin | Gentisic acid ^{l} | I-alanine ^{l} |
| Arginine | a CMPF-related metabolite ^{l} | Sarcosine ^{l} |
| Glycylvaline | DHEA sulfate ^{l} | Proline betaine ^{l} |

^{l} from para. [0076] in US 2019/0178900, which is incorporated herein by reference.

Other suitable examples of additional ASD-related metabolites include the metabolites listed in Table 6 of PCT Application No. PCT/US2014/045397, the relevant contents being incorporated herein by reference.

The method described herein further comprises step (c) comparing the concentration levels of the ASD-related metabolites from the obtained sample to the concentration levels of reference ASD-related metabolites from an ASD-negative sample. One skilled in the art will appreciate that references can be established as a value representative of the level of ASD-related metabolites in a non-autistic population that do not suffer from ASD for the comparison. Various criteria may be used to determine the inclusion and/or exclusion of a particular subject in the reference population, including age of the subject (e.g., the reference subject can be within the same age group as the subject in need of treatment) and gender of the subject (e.g., the reference subject can be the same gender as the subject in need of treatment). In certain embodiments, the reference is from an ASD-negative sample obtained from a non-autistic child aged about 10 years or less, about 5 years or less, about 3 years or less or about 18 months or less. In certain embodiments, with the methods described herein, the subject is a child aged about 10 years or less, about 5 years or less, about 3 years or less or about 18 months or less.

The method described herein further comprises step (d) identifying the subject as having ASD if the concentration levels of the ASD-related metabolites from the obtained sample are different relative to the concentration levels of the reference ASD-related metabolites from the ASD-negative sample. In certain embodiments, the identifying step (d) occurs upon determination that the concentration level of the at least one ASD-related metabolite from the obtained sample differs by about 20% or more, about 30% or more, about 40% or more, about 50% or more, about 60% or more, or about 70% or more relative to the concentration level of the at least one reference ASD-related metabolite from the ASD-negative sample. In certain embodiments, the identifying step (d) occurs upon determination that the concentration levels of at least two, at least three, at least four or at least five ASD-related metabolites from the obtained sample differ by about 20% or more, about 30% or more, about 40% or more, about 50% or more, about 60% or more, or about 70% or more relative to the concentration levels of the reference ASD-related metabolites from the ASD-negative sample.

In certain embodiments, the identifying step (d) occurs upon determination that the concentration levels of the fumaric acid and/or the L-malic acid from the obtained sample are decreased relative to the concentration levels of the reference fumaric acid and/or the reference L-malic acid from the ASD-negative sample. In some aspects, the concentration level of the fumaric acid level from the obtained sample is lower than about 20% or more, about 30% or more, about 40% or more, about 50% or more, about 60% or more, or about 70% or more relative to the concentration level of the reference fumaric acid from the ASD-negative sample. In some aspects, the concentration level of the L-malic acid level from the obtained sample is lower than about 20% or more, about 30% or more, about 40% or more, about 50% or more, about 60% or more, or about 70% or more relative to the concentration level of the reference fumaric acid from the ASD-negative sample.

In certain embodiments, the identifying step (d) occurs upon determination that the concentration level of the 3-(3-Hydroxyphenyl)-3-hydroxypropanoic acid (HPHPA) from the obtained sample is increased relative to the concentration level of the reference 3-(3-Hydroxyphenyl)-3-hydroxypropanoic acid (HPHPA) from the ASD-negative sample. In some aspects, the concentration level of the 3-(3-Hydroxyphenyl)-3-hydroxypropanoic acid (HPHPA) from the obtained sample is elevated by about 20% or more, about 30% or more, about 40% or more, about 50% or more, about 60% or more, or about 70% or more relative to the concentration level of the reference 3-(3-Hydroxyphenyl)-3-hydroxypropanoic acid (HPHPA) from the ASD-negative sample. In some aspects, an elevated 3-(3-Hydroxyphenyl)-3-hydroxypropanoic acid (HPHPA) level of about 100 umol/mmol creatinine or more places identifies the subject as having ASD. In some aspects, a 3-(3-Hydroxyphenyl)-3-hydroxypropanoic acid (HPHPA) level of less than about 100 umol/mmol creatinine excludes the subject from being identified as having ASD.

The method described herein further comprises step (e) treating the subject so identified as having ASD with an ASD treatment regime.

In certain embodiments, with any of the methods described herein, the comparison of the concentration level of the at least one ASD-related metabolite from the obtained sample to the concentration level of the reference ASD-related metabolite from the ASD-negative sample comprises using multivariate statistical analysis. Preferably, the multivariate statistical analysis is selected from principal component analysis (“PCA”), or partial least squares projects to latent structures discriminant analysis (“PLS-DA”). In certain embodiments, a computer is used for statistical analysis. Data for statistical analysis can be extracted from chromatograms (i.e., spectra of mass signals) using software for statistical methods known in the art.

In some aspects, the present disclosure relates to a method of monitoring ASD progression and treating the ASD in a subject. Essentially the method includes quantifying the ASD-related metabolites at one or more time points after the initiation of treatment to monitor ASD progression (e.g., rate of decline or rate of improvement of ASD progression) in a subject. Accordingly, the method comprises: (a) providing a first biological sample obtained from the subject at a first time; (b) assessing a first ASD-related metabolite profile by measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the first obtained sample; (c) comparing the first ASD-related metabolite profile with a reference ASD-related metabolite profile from an ASD-negative sample; (d) determining that there is a first difference between the first ASD-related metabolite profile and the reference ASD-related metabolite profile from the ASD-negative sample, the first difference being indicative of ASD; (e) providing a second biological sample obtained from the subject at a second time that is after the first time; (f) assessing a second ASD-related metabolite profile by measuring concentration levels of the ASD-related metabolites from the second obtained sample; (g) comparing the second ASD-related metabolite profile with the reference ASD-related metabolite profile from the ASD-negative sample; (h) determining that there is a second difference between the first ASD-related metabolite profile and the reference ASD-related metabolite profile from the ASD-negative sample, the second difference being indicative of ASD; (i) determining ASD progression based on at least in part on the first and second difference; and (j) treating the subject as identified with an ASD treatment regime.

In certain embodiments of the above method, the period between the first time and the second time is at least 1 month, at least 2 months, at least 3 months, at least 6 months, at least 9 months, or at least 12 months, preferably at least 3 months. In some embodiments, the treatment has been administered to the subject before the first two biological samples have been obtained. In other embodiments, the treatment has been administered to the subject in the interval(s) between the taking of the biological samples. In certain embodiments, the first biological sample, the second biological sample, or both are blood or urine, preferably serum, plasma or urine.

In certain embodiments of the above method, the determining step (i) occurs upon determination that the concentration levels of the acylcarnitine, preferably selected from C10:1, C16:2 and/or C7-DC, from the second biological sample are increased relative to the concentration levels of the C10:1, the C16:2 and/or the C7-DC from the first obtained biological sample.

In certain embodiments of the above method, the determining step (i) occurs upon determination that the concentration levels of the fumaric acid and/or the L-malic acid from the second obtained biological sample are decreased relative to the concentration levels of the fumaric acid and/or the L-malic acid from the first obtained biological sample.

In certain embodiments of the above method, the determining step (i) occurs upon determination that the concentration level of the 3-(3-Hydroxyphenyl)-3-hydroxypropanoic acid (HPHPA) from the second obtained biological sample is increased relative to the concentration level of the 3-(3-Hydroxyphenyl)-3-hydroxypropanoic acid (HPHPA) from the first obtained biological sample.

In certain embodiments of the above method, the determining step (i) occurs upon determination that the concentration levels of the lysoPC a C17:0 and/or the lysoPC a C20:3 from the second obtained biological sample are increased related to the concentration levels of the lysoPC a C17:0 and/or the lysoPC a C20:3 from the first obtained biological sample.

In certain embodiments of the above method, the determining step (i) occurs upon determination that the concentration levels of the SM (OH) C24:1 and/or the SM (OH) C22:2 from the second obtained biological sample SM (OH) C24:1 and/or the SM (OH) C22:2 from the obtained sample SM (OH) C24:1 and/or the SM (OH) C22:2 from the second obtained biological sample.

In certain embodiments of the above method, the determining step (i) occurs upon determination that the concentration levels of the PC ae C36:0 and/or the PC aa C40:2 from the second obtained biological sample are increased relative to the concentration levels of the PC ae C36:0 and/or the PC aa C40:2 from the first obtained biological sample.

In certain embodiments of the above method, the determining step (i) occurs upon determination that the concentration levels of the SM (OH) C24:1 and/or the SM (OH) C22:2 from the second obtained biological sample SM (OH) C24:1 and/or the SM (OH) C22:2 from the obtained sample SM (OH) C24:1 and/or the SM (OH) C22:2 from the second obtained biological sample.

In certain embodiments of the above method, the determining step (i) occurs upon determination that the concentration levels of the PC ae C36:0 and/or the PC aa C40:2 from the second obtained biological sample are increased relative to the concentration levels of the PC ae C36:0 and/or the PC aa C40:2 from the first obtained biological sample.

In certain embodiments of the above method, the determining step (i) occurs upon determination that the concentration levels of the 4-hydroxymandelic acid and/or the 2-hydroxyisovaleric acid from the second obtained biological sample are decreased relative to the concentration levels of the 4-hydroxymandelic acid and/or the 2-hydroxyisovaleric acid from the first obtained biological sample.

In certain embodiments of the above method, the determining step (i) occurs upon determination that the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the second obtained biological sample are increased relative to the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the first obtained biological sample.

The present disclosure also provides for a method for diagnosing and treating Autism Spectrum Disorder (ASD) in a subject. The method comprises: (a) providing a biological sample obtained from the subject; (b) measuring concentration levels of at least one, at least two, at least three, at least four or at least five ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C; (c) comparing the concentration levels of the ASD-related proteins from the obtained sample to the concentration levels of reference ASD-related proteins from an ASD-negative sample; (d) identifying the subject as having ASD if the concentration levels of the ASD-related proteins from the obtained sample are different relative to the concentration levels of the reference ASD-related proteins from the ASD-negative sample; and (e) treating the subject so identified with an ASD treatment regime.

The present disclosure also provides for a method for diagnosing and treating Autism Spectrum Disorder (ASD) in a subject. The method comprises: (a) providing a biological sample obtained from the subject; (b) measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample; (c) comparing the concentration levels of the ASD-related metabolites from the obtained sample to the concentration levels of reference ASD-related metabolites from an ASD-negative sample; (d) measuring concentration levels of at least one, at least two, at least three, at least four or at least five ASD-related proteins selected from the group consisting of retinol-binding protein 4 (RBP4), apolipoprotein A-II, serotransferrin, thrombospondin-1 (TSP-1), coagulation factor XIII A chain, alpha-2-antiplasmin, coagulation factor X, coagulation factor XI, alpha-1-antitrypsin, insulin-like growth factor-binding protein 2 and tenascin C; (e) comparing the concentration levels of the ASD-related proteins from the obtained sample to the concentration levels of reference ASD-related proteins from an ASD-negative sample; (f) identifying the subject as having ASD if the concentration levels of the ASD-related metabolites from the obtained sample are different relative to the concentration levels of the reference ASD-related metabolites from the ASD-negative sample, and the concentration levels of the ASD-related proteins from the obtained sample are different relative to the concentration levels of the reference ASD-related proteins from the ASD-negative sample; and (g) treating the subject so identified with an ASD treatment regime.

In certain embodiments of the above method, the identifying step (f) occurs upon determination that the concentration levels of at least one, at least two, at least three, at least four or at least five of the ASD-related proteins from the obtained sample differ by about 20% or more, about 30% or more, about 40% or more, about 50% or more, about 60% or more or about 70% or more relative to the concentration levels of the reference ASD-related proteins from the ASD-negative sample.

In certain embodiments of the above method, the identifying step (f) occurs upon determination that the concentration levels of alpha-2-antiplasmin, coagulation factor X, coagulation factor XI and/or tenascin C from the obtained sample are increased relative to the concentration levels of the reference alpha-2-antiplasmin, the reference coagulation factor X, the reference coagulation factor XI and/or the reference tenascin C from the ASD-negative sample.

In certain embodiments of the above method, the identifying step (f) occurs upon determination that the concentration levels of coagulation factor XIII A chain, thrombospondin-1 (TSP-1) and/or retinol-binding protein 4 (RBP4) are decreased relative to the concentration levels of the reference coagulation factor XIII A chain, the reference thrombospondin-1 (TSP-1) and/or the reference retinol-binding protein 4 (RBP4) from the ASD-negative sample.

In certain embodiments of the above method, the identifying step occurs upon determination that the concentration levels of at least one, at least two, at least three, at least four or at least five of the ASD-related metabolites from the obtained sample differ by about 20% or more, about 30% or more, about 40% or more, about 50% or more, about 60% or more or about 70% or more relative to the concentration levels of the reference ASD-related metabolites from the ASD-negative sample.

In certain embodiments of the above method, the identifying step (f) occurs upon determination that the concentration levels of 3-(3-Hydroxyphenyl)-3-hydroxypropanoic acid (HPHPA), acylcarnintine, lysophospholipid, sphingolipid and/or glycerophospholipid from the obtained sample are increased relative to the concentration levels of the reference 3-(3-Hydroxyphenyl)-3-hydroxypropanoic acid (HPHPA), the reference acylcarnintine, the reference lysophospholipid, the reference sphingolipid and/or the reference glycerophospholipid from the ASD-negative sample.

In certain embodiments of any of the above methods, the acylcarnitine is selected from C10:1, C16:2 and/or C7-DC. Preferably, the identifying step of the method occurs upon determination that the concentration levels of the C10:1, the C16:2 and/or the C7-DC from the obtained sample are increased relative to the concentration levels of the reference C10:1, the reference C16:2 and/or the reference C7-DC from the ASD-negative sample.

In certain embodiments of any of the above methods, the lysophospholipid is lysoPC a C17:0, and/or lysoPC a C20:3. Preferably, the identifying step of the method occurs upon determination that the concentration levels of the lysoPC a C17:0 and/or the lysoPC a C20:3 from the obtained sample are increased relative to the concentration levels of the reference lysoPC a C17:0 and/or the reference lysoPC a C20:3 from the ASD-negative sample.

In certain embodiments of any of the above methods, the sphingolipid is SM (OH) C24:1 and/or SM (OH) C22:2. Preferably, the identifying step of the method occurs upon determination that the concentration levels of the SM (OH) C24:1 and/or the SM (OH) C22:2 from the obtained sample are increased relative to the concentration levels of the reference SM (OH) C24:1 and/or the reference SM (OH) C22:2 from the ASD-negative sample.

In certain embodiments of any of the above methods, the glycerophospholipid is PC ae C36:0 and/or PC aa C40:2. Preferably, the identifying step of the method occurs upon determination that the concentration levels of the PC ae C36:0 and/or the PC aa C40:2 from the obtained sample are increased relative to the concentration levels of the reference PC ae C36:0 and/or the reference PC aa C40:2 from the ASD-negative sample.

In certain embodiments of any of the above methods, the identifying step occurs upon determination that the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the obtained sample are increased relative to the concentration levels of the reference p-hydroxyphenylacetic acid, 2-ethyl-3-hydroxypropionic acid, 3-methylglutaconic acid, 3-hydroxyisovaleric acid, 3-methyl glutaric acid, and/or 4-hydroxyhippuric acid from the ASD-negative sample.

The ASD treatment regime is selected from the group consisting of dietary adjustment, nutritional supplement, behavior training or a combination thereof, to the subject diagnosed as having or predisposed of developing the ASD. Preferably, the ASD treatment regime has the effect of adjusting the concentration levels of one or more of the ASD-related metabolites in the subject diagnosed as having or predisposed of developing the ASD towards the corresponding levels of the reference ASD-related metabolites from the ASD-negative sample.

Various methods can be used to adjust the diet of the subject. For non-limiting example, a reduced carbohydrate diet can be provided to the subject to reduce one or more intestinal bacterial species. Without wishing to be bound by theory, it is believed that a reduced carbohydrate diet can restrict the available material for fermentation and decrease the composition of gut microbiota in the subject. For instance, it may be desirable to reduce the level of Clostridia bacteria (such as Lachnospiraceae) in the subject to treat ASD. Moreover, the induction of increased fatty acid oxidation and reduced blood glucose level through ketogenic diet may improve symptoms in subjects, particular children, with ASD.

The nutritional supplement can be adjusted, for non-limiting example, by fortified food or dietary supplement that provides health benefits to the subject. As used herein, the term “probiotic” generally refers to live microorganisms, which, when administered in adequate amounts, confer a health benefit, and may help to treat ASD in the subject. The probiotics may be available in fortified food and nutritional supplements (e.g., capsules, tablets, and powders). Non-limiting examples of fortified food containing probiotic include dairy products such as yogurt, fermented and unfermented milk, smoothies, butter, cream, hummus, kombucha, salad dressing, miso, tempeh, nutrition bars, and some juices and soy beverages.

The behavior training refers to any activity that reduces the burden of the individual expressing or later developing those behavioral symptoms associated with ASD. Behavior training may include standard behavioral modification treatments as is generally known in the art.

Various methods can be used to adjust the concentration level, for example blood level (e.g., serum level), of the ASD-related metabolite in the subject. Preferably, the adjustment of the concentration level of the one or more ASD-related metabolites in the subject occurs until an improvement in the behavioral performance in the subject is observed.

In certain embodiments, an antibody that specifically binds the ASD-related metabolite, an intermediate for the in vivo synthesis of the ASD-related metabolite, or a substrate for the in vivo synthesis of the ASD-related metabolite can be administered to the subject. For example, an antibody that specifically binds HPHPA and/or one or more of the substrates and intermediates in the in vivo HPHPA synthesis can be used to reduce the level of HPHPA in the subject.

In certain embodiments, the concentration level, for example blood level (e.g., serum level), of the one or more ASD-related metabolites is adjusted by adjusting the composition of gut microbiota in the subject. In certain embodiments, adjusting the composition of the gut microbiota in the subject includes increasing the levels of one or more bacterial species in the subject. For non-limiting example, the levels of Ruminococcaeceae, Erysipelotrichaceae, and/or Alcaligenaceae bacteria can be increased to adjust the composition of the gut microbiota in the subject. In certain embodiments, adjusting the composition of the gut microbiota in the subject includes decreasing the levels of one or more bacterial species in the subject. For non-limiting example, the level of Clostridia bacteria can be decreased to adjust the composition of the gut microbiota in the subject.

In certain embodiments, the composition of gut microbiota in the subject is adjusted by fecal transplantation (known as fecal microbiota transplantation (“FMT”), fecal bacteriotherapy or stool transplant). Fecal transplantation can include a process of single or multiple transplantation of fecal bacteria from a healthy donor (i.e., ASD-negative individual) to a recipient (e.g., subject suffering from ASD).

In certain embodiments, the composition of gut microbiota in the subject is adjusted by administration of a composition comprising bacteria to the subject, for example a composition comprising Bacteroides bacteria (e.g., B. fragilis). The bacterial composition can be administered to the subject via oral administration, rectum administration, transdermal administration, intranasal administration or inhalation. With oral administration, the bacterial composition can be a probiotic composition, a nutraceutical, a pharmaceutical composition or a mixture thereof. The dosage for human and animal subjects preferably contains a predetermined quantity of the bacteria calculated in an amount sufficient to produce the desired effect.

### Kits

The metabolomic profile described herein may be utilized in tests, assays, methods, kits for diagnosing, predicting, modulating or monitoring ASD, including ongoing assessment, monitoring, susceptibility assessment, carrier testing and prenatal diagnosis. The present disclosure includes a kit for diagnosis of ASD by measuring and identifying at least one or more ASD-related metabolites associated with ASD. Preferably, the kit may comprise appropriate ASD treatment regime to be initiated upon the determination of ASD. Accordingly, the kit comprises (a) a detector configured to detect concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from an obtained biological sample, (b) a composition comprising fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose in control levels corresponding to control group of ASD-negative subjects, (c) a multivariate analysis system configured to analyze a difference in the concentration levels of the ASD-related metabolites and the control levels, and (d) optionally, instruction for an ASD diagnosis method, wherein the method comprises measuring, using the detector, the levels of the ASD-related metabolites from the obtained biological sample, and comparing the levels of the obtained ASD-related metabolites to the control levels of the ASD-related metabolites obtained from ASD-negative subjects. Preferably, the ASD diagnosis method comprises a multi-metabolite detector configured to measure the levels of ASD-related metabolites.

In some aspects, the kit may be for the measurement of the ASD-related metabolites by a physical separation technique (as described herein above). In some aspects, the kit may be for measurement of the ASD-related metabolites by a methodology other than a physical separation method, such as for non-limiting example, a colorimetric, enzymatic, and immunological methodology. The kit may also include one or more appropriate negative and/or positive controls. Kit of the present disclosure may include other reagents such as buffers and solutions needed to perform the tests.

### Computer-Implemented Method

The disclosure is also directed to a computer-implemented method for processing a biological sample of a subject, diagnosing an ASD and treating the ASD. The computer-implemented method may further allow monitoring of ASD progression across multiple time points to support more effective treatment regime.

The computer-implemented method comprises receiving a biological sample from the subject; processing the sample in a spectroscopy unit directly or wirelessly linked, or may utilize any suitable communication technology, to a processing device, the processing device having memory for storing measurement data from the spectroscopy unit; and in the spectroscopy unit, measuring levels of least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose and storing the measurement data in the processor. The processing device comprises one or more data storage devices that may be configured or adapted to store data related to the method. For example, the data storage device may be configured or adapted to store measurement data from the spectroscopy unit. The data storage device may also comprise computer program code stored thereon. The program code of this embodiment may include program code for at least performing the steps of the method aspect upon execution thereof.

The computer-implemented method further comprises comparing the stored measurement data to a value in the memory representing an ASD-negative sample using multivariate statistical analysis; storing on the processing device a result corresponding to at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample, wherein the result identifies the subject as having ASD if the measurement data representing the level of the ASD-related metabolite is different relative to a concentration value of a reference ASD-related metabolite from an ASD-negative sample; and displaying an ASD treatment regime on an electronic display connected directly or wirelessly to the processor for the subject identified as having ASD or as having predisposition of developing ASD. The displayed treatment regime comprises electronic text, optionally with graphical icons, on a graphical user interface describing one or more of: dietary adjustments, nutritional supplements, behavior training or a combination thereof, to the subject diagnosed as having or predisposed of developing the ASD, or adjusting the blood levels of one or more of the ASD-related metabolites in the subject diagnosed as having or predisposed of developing the ASD until an improvement in the behavioral performance in the subject is observed, preferably the adjustment of the blood levels of one or more of the ASD-related metabolites comprises adjusting the composition of gut microbiota in the subject.

### EXAMPLES

The following examples describe some exemplary modes of practicing certain methods that are described herein. It should be understood that the examples are for illustrative purposes only and are not meant to limit the scope of the systems and methods described herein.

### Example 1

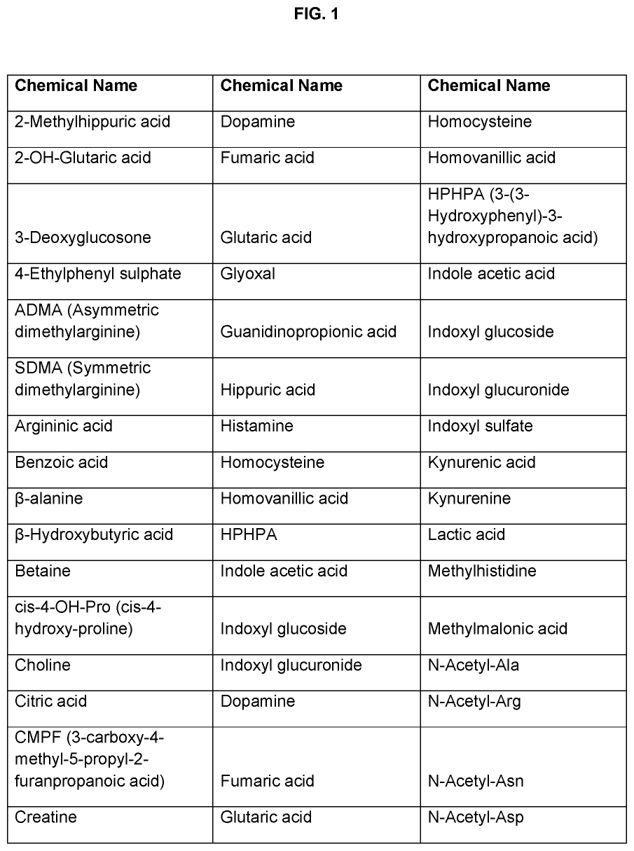

Urine samples from subjects with ASD and ASD-negative control subjects, ages 2 to 18 years, are included in this study. Single spot urine samples are acquired from the subjects and analyzed via GC-MS with quantitative detection of up to 85 urine organic acids (see FIG. 1). The urine samples are stored at −20° C. until thawed for analysis. The intervals between collection and analysis range from 2 weeks to 2 months.

### Preparation of the Samples for GC-MS Analysis

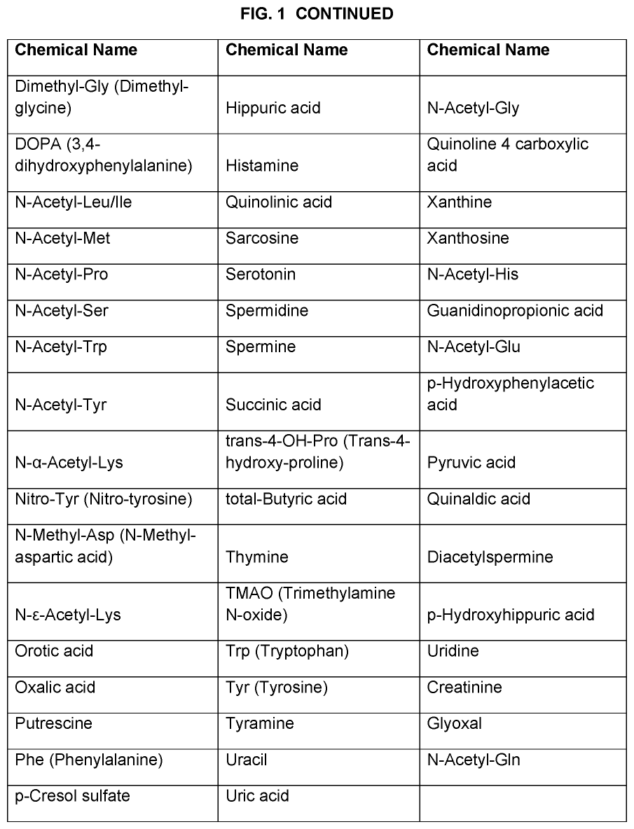

To prepare the samples, 200 μL of HPLC water, 10 μL of internal standards (ISTD) (2.82 mM tropic acid as internal standard), and 40 μL of methoxyamine HCl are combined in a 2 mL glass vial and mixed thoroughly. The samples are incubated at 60° C. for 30 minutes. The samples are cooled to room temperature for 10 mins on the bench and subsequently, 10 μL of 6 N HCl is added to the samples until the solution is acidic (pH=1). A volume of 210 μL of blank/QC/urine samples is transferred from the vial to 2 mL Eppendorf™ tubes. Ethyl acetate is added (600 μL) to the tubes and the resultant solution is vortexed thoroughly for 1 minute. The samples are sprung at 10,000 rpm for 3 mins. A volume of 500 μL of the supernatant is transferred into a new 2 mL glass vial. Subsequently, another 600 μL of ethyl acetate is added to the Eppendorf™ tubes and vortexed thoroughly for 1 min. The samples are sprung at 10,000 rpm for 3 mins. A volume of 500 μL of the supernatant is withdrawn, and added into the 2 mL glass vial containing the previous supernatant (i.e., combine the supernatant from the two extractions). The samples are evaporated to dryness under nitrogen with heat (35° C.) for 1.5 hrs. A volume of 160 μL of hexane and 40 μL of fresh N,O-Bis(trimethylsilyl)trifluoroacetamide (BSFTA) (with 1% N,O-Bis(trimethylsilyl)trifluoroacetamide (TMCS)) are added to the sample, and the resultant mixture vortexed to mix thoroughly. The samples are incubated at 80° C. for 30 mins. After incubation, the samples are cooled to room temperature for 15 mins on the bench. A volume of 190 μL of the samples is transferred to 250 μL inserts for blank, QCs, and urine samples. An alkane standard mixture was prepared fresh, i.e., 25 μL of alkane standard solution C₈-C₂₀ and 75 μL of alkane standard solution C₂₁-C₄₀ are added into a 2 mL glass vial, vortexed to mix thoroughly, and transferred to 250 μL insert. The samples are ready for GC-MS analysis or are refrigerated until ready for analysis. See FIG. 2 for a flowchart of the GC-MS and analysis process.

### GC-MS Data Analysis

The GC-MS data which contains fully quantified metabolite concentration data for nearly 85 organic acids are analyzed via MetaboAnalyst™ V3.0 (2016), a comprehensive web-based server that can perform statistical, functional, and integrative analysis of quantified metabolite datasets. MetaboAnalyst™ tools are used to generate specific data calculations and visualizations to identify metabolites that are used to classify ASD versus healthy control sample.

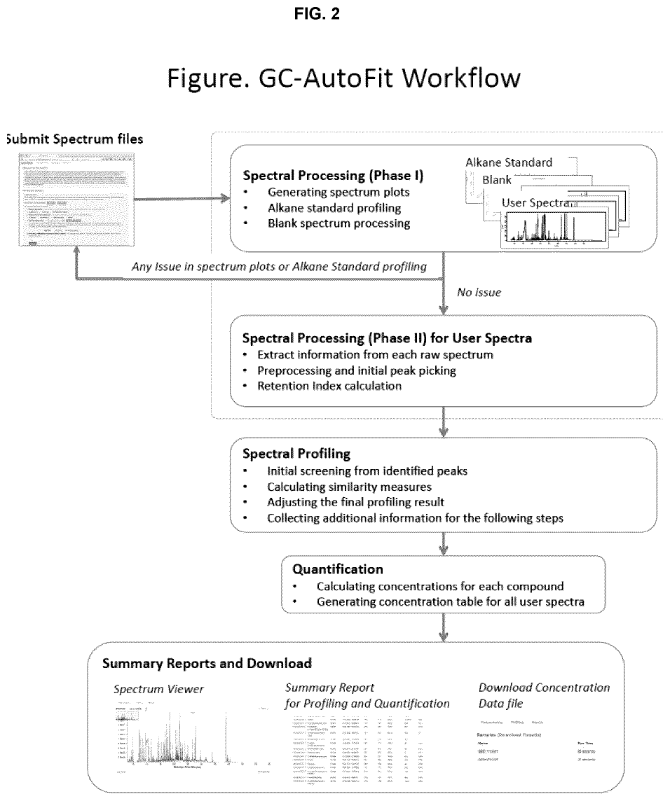

A principal component analysis (“PCA”) and Partial Least Squares Discriminant Analysis (“PLS-DA”) are carried out for the ASD and control groups and results are compared. PCA is a multivariate clustering technique used to visualize differences in sample populations and to bring out strong clusters or patterns in datasets. Applicant used the multivariate technique to make the data easier to analyze and visualize. MetaboAnalyst™ is used to generate a PCA plot and start to visualize clusters of ASD samples and healthy control samples (see FIG. 3). PLS-DA is another classification model that can be performed to enhance the separation between the two groups of observations, by rotating PCA components such that a maximum separation among classes is obtained. PLS-DA can also be used to understand and rank which variables carry the most significant class separating information. PLS-DA allows for the enhancement of separation and classification of ASD versus healthy control populations (see FIG. 3).

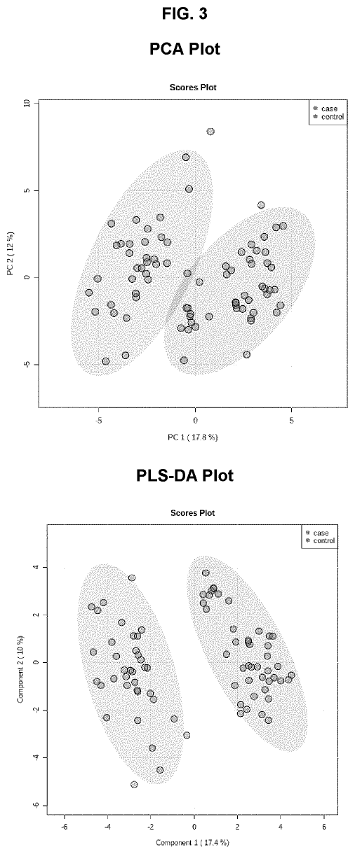

A receiver operating characteristic curve, or ROC curve is generated using MetaboAnalyst™ (see FIG. 4). A ROC curve is a graphical plot that illustrates the diagnostic ability of a binary classifier system as its discrimination threshold is varied. By computing the area under the ROC curve (AUC), it is possible to determine that the diagnostic ability for the model to discriminate between ASD versus healthy control samples is 0.986. This corresponds to a level of 98.6% diagnostic accuracy.

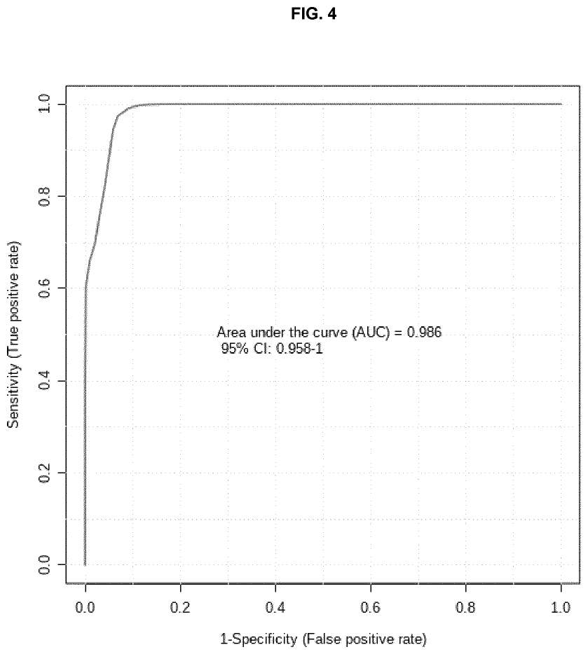

With reference to FIG. 5, a Variable Importance of Projection (“VIP”) plot is generated to identify the most significant variables (i.e., metabolites) in descending order where the top variables contribute more to the PLS-DA model than the bottom ones. Those at the top of the VIP plot also have high predictive power in classifying between the ASD and healthy control urine samples.

### Example 2

Fasting blood samples are collected by venipuncture from ASD subjects (i.e., the Test Group). The Test Group includes 44 participants between the ages of 3 and 32 years and 76% of whom are male. For comparative analysis, 40 non-ASD subjects (i.e., the Control Group) are randomly selected from a database created by the Applicant (herein “Molecular You database”). The Control Group are age- and sex-matched, with most of the Control Group around 9 years of age and approximately 50% of whom are male. The demographics for the Test and Control Groups are provided in Table 2.

TABLE 2
Subject Demographics

| | | Age | Median | Max | Min | |
|---|---|---|---|---|---|---|
| Group | Sex | Category | Age | Age | Age | n |
| Control | Female | Adult | 32 | 32 | 22 | 3 |
| Control | Female | Child | 9 | 10 | 8 | 19 |
| Control | Male | Adult | 32 | 32 | 32 | 1 |
| Control | Male | Child | 9 | 16 | 9 | 17 |
| Test | Female | Child | 9 | 15 | 3 | 11 |
| Test | Male | Adult | 21 | 32 | 18 | 6 |
| Test | Male | Child | 10 | 16 | 3 | 27 |

To produce the serums, the blood samples are collected into 6 mL serum vacutainer tubes, protected from light, and incubated for 1 hr at room temperature post-draw to allow clotting. After incubation, the blood samples are centrifuged at 1,300×g for 10 mins to isolate the serum. The serums are transferred to cryovials and immediately stored at −20° C. until thawed for analysis. The intervals between collection and analysis range from 2 weeks to 2 months.

To produce the plasma, the blood samples are collected into 6 mL EDTA vacutainer tubes and immediately placed into an ice bath post-draw. After the ice bath, the blood samples are centrifuged at 1,300×g for 10 mins to isolate the plasma. The plasma is transferred to cryovials and immediately stored at −20° C. until thawed for analysis. The intervals between collection and analysis range from 2 weeks to 2 months.

### (A) Proteomics Analysis

Peptides are synthesized using fluorenylmethoxycarbonyl (FMOC) chemistry with 13C/15N-labeled amino acids for stable isotope labeled standard (SIS)-peptides, purified through reverse phase-HPLC with subsequent assessment by MALDI-TOF-MS, and characterized via amino acid analysis (AAA) and capillary zone electrophoresis (CZE). All other chemicals and reagents employed are of the highest analytical and LC-MS grade available obtained from commercial vendors.

A panel of 140 proteins are used for targeted quantitation by peptide-based analysis using MC-MRM mass spectrometry. These peptides have been previously validated for their use in LC-MRM experiments following the National Cancer Institute's Clinical Proteomic Tumor Analysis Consortium (CPTAC) guidelines for assay development (https://asays.cancer.gov/).

### Preparation of the Samples for Proteomic Analysis

The frozen plasmas are thawed at room temperature. A volume of 10 μL of the plasma is subjected to 9 M urea, 20 mM dithiothreitol, and 0.5 M iodoacetamide, sequentially. All steps are carried out in Tris buffer at pH 8.0. Denaturation and reduction occurred simultaneously at 37° C. for 30 mins, with alkylation occurring thereafter in the dark, at room temperature for 30 mins. Proteolysis is initiated by the addition of TPCK-treated trypsin (70 μL at 1 mg/mL; Worthington) at a 10:1 substrate:enzyme ratio. After overnight incubation at 37° C., proteolysis is quenched with formic acid (FA) at a final concentration of 1.0%.

The SIS peptide mixture (as described above) is then spiked into the plasma. All plasma are then concentrated by solid-phase extraction (2 mg of Water's Oasis® HLB (30 micron sorbent particles)). After solid-phase extraction, the concentrated eluate is evaporated using a speed vacuum concentrator, and rehydrated in 0.1% FA to a final concentration of 1 μg peptides/4 digest for LC-MRM/MS. A surrogate matrix for use with standard and QC samples is prepared from a digest of 10 mg/mL BSA in PBS buffer, using the same methodology as plasma samples described above.

The standard curves are generated using a natural isotopic abundance (NAT) peptide for each analyte. A dilution series of the NAT peptides in the surrogate matrix is prepared from a high concentration of 1000× the lower limit of quantitation (LLOQ) over 8 dilutions to the lowest point of the curve, which is also the LLOQ for the assay. The QC samples are prepared from the same NAT mix and diluted in BSA digest at 4×, 50×, and 500× the LLOQ for each peptide.

Injections of 10 μL of the plasma tryptic digests are separated with a Zorbax Eclipse Plus RP-UHPLC™ column (2.1×150 mm, 1.8 μm particle diameter; Agilent) that is contained within a 1290 Infinity system (Agilent™). Peptide separations are achieved at 0.4 mL/min over a 60 min run, via a multi-step LC gradient (2-80% mobile phase B; mobile phase compositions: A was 0.1% FA in H₂O while B was 0.1% FA in acetonitrile). The column is maintained at 50° C. A post-gradient column re-equilibration of 4 mins is used after each sample analysis. The LC system is interfaced to a triple quadrupole mass spectrometer (Agilent™ 6495B) via a standard-flow AJS ESI source, operated in the positive ion mode.

### Quantitative Analysis

The MRM data is visualized and examined with Skyline Quantitative Analysis™ software (version 4.2.0.19072, University of Washington). This involves peak inspection to ensure accurate selection, integration, and uniformity (in terms of peak shape and retention time) of the SIS and NAT peptides. After defining a small number of criteria (i.e., 1/x2 regression weighting, <20% deviation in the QC's level's accuracy) the standard curve is used to calculate the peptide concentration in fmol/μL of plasma in the samples through linear regression.

The following criteria are applied to accept a batch:
1. 5 of the 8 standards with back-calculated concentrations within ±20% of theoretical concentration.
2. At least 66% of QC samples within ±20% of theoretical concentration.
3. 95% of peptide calibration curves acceptable.
4. For the pooled human plasma sample QC, ⅔ of all peptides should be within 30% of the mean concentration determined during analysis.
5. Chromatographic peaks are manually inspected for retention time consistency, peak shape and absence interferences.

### (B) Metabolomics Analysis

The method is based on LC-MS/MS in a positive scheduled multiple reaction monitoring (MRM) mode and direct infusion (DI) MS/MS mode for detection and quantifying metabolites in serum in a 96-well format. The assay has two different derivation methods (e.g., a pre-column amine derivatization step using phenylisothiocyanate) and three different runs (LC-MS/MS and DI MS/MS) for different classes of metabolites. Classes of Metabolites include:
1. Amino acid and biogenic amines
2. Organic acids
3. Acylcarnitines
4. Lipids
5. Glucose
6. Others

The MS data analysis is performed, and concentrations are calculated using Analyst 1.6.2. (SCIEX).

The following criteria are applied to accept a batch:
1. Calibration curve with R2>0.99.
2. All QC standard samples within 0% of the theoretical concentrations (not applicable to acylcarnitines and phospholipids).
3. From the NIST human serum QC sample, calculated concentrations should be within 15% of the known concentrations or the mean concentrations determined during analysis.

### (C) Exposomic Analysis

The method is based on LC-MS/MS in a positive scheduled multiple reaction monitoring (MRM) mode and inductively coupled plasma (ICP)-MS for detection and quantifying metabolites in serum. The assay has two different derivation methods (e.g., a pre-column amine derivatization step using phenylisothiocyanate), three LC-MS/MS, and one ICP-MS run for different classes of metabolites.

The following criteria are applied to accept a batch:
1. Calibration curve with R2>0.99 (applies to ICP-MS as well).
2. All QC standard samples within 20% of the theoretical concentrations (not applicable to acylcarnitines and phospholipids).
3. From the NIST human serum QC sample, calculated concentrations should be within 15% of the known concentrations or the mean concentrations determined during analysis.

### (D) Data Analysis

Data wrangling and analysis are performed in R (3.5.1) and MetaboAnalyst™ (https://www.metaboanalyst.ca/).

The concentration after absolute quantification is used for all metabolite-related analyses, while protein concentrations are converted from peptide values. Additionally, for any analytes with concentrations below the limit of detection (LOD) or the limit of quantification (LLOQ) of the analytical instruments, half of the analytical LOD is assigned as the concentration. The proportion of samples that are below LOD or LLOQ are calculated per analyte in each group (Test and Control Groups). Analytes with >30% difference between the proportion of LODs/LLOQs are filtered from analysis in order to avoid misrepresentations in comparison analyses or false significant findings for univariate comparison of means. After this process, there are 80 metabolites and 131 proteins remaining in the data.

The data is normalized using a generalized logarithm transformation and scaled (mean-centred and divided by the standard deviation of each variable).

Using the reference range, analytes are grouped based on whether they fall within the reference range, within 10% of the reference range boundaries (±5% on each side of the reference range cut-off values), and outside of the reference range (>5% of the reference range cut-off values). Analytes that are non-detectable in the data are classified as “ND”. If no corresponding age- or sex-specific reference range values are available in Molecular You database, the data points are classified as “NA”.

### (E) Results

The analyte concentration between the Test and Control Groups are compared using a Wilcoxon rank-sum test. Results show that 41 out of 80 metabolite concentrations are significantly different between the Test and Control Groups after adjusting for the false-discovery rate (holm's method) of multiple comparisons. The 10 metabolites with the largest absolute effect between the Test and Control Groups are shown in Table 3.

TABLE 3
Ten Metabolites with Largest Effect

| | Measure | Control | ASD | Effect | |
|---|---|---|---|---|---|
| Metabolite | Category | (n) | (n) | Size | p |
| C10:1 | Acylcarnitine | 40 | 44 | 1.40 | 9.17e−09 |
| C16:2 | Acylcarnitine | 40 | 44 | 1.30 | 9.48e−08 |
| Glucose | Sugar | 40 | 44 | 1.26 | 1.32e−07 |
| lysoPC a | Lysophospholipid | 40 | 44 | 1.25 | 1.79e−08 |
| C17:0 | | | | | |
| SM (OH) | Sphingolipid | 40 | 44 | 1.14 | 6.64e−07 |
| C24:1 | | | | | |
| PC ae | Glycerophospholipid | 40 | 44 | 1.11 | 7.81e−08 |
| C36:0 | | | | | |
| lysoPC a | Lysophospholipid | 40 | 44 | 1.09 | 1.48e−05 |
| C20:3 | | | | | |
| PC aa | Glycerophospholipid | 40 | 44 | 1.00 | 5.16e−10 |
| C40:2 | | | | | |
| SM (OH) | Sphingolipid | 40 | 44 | 0.97 | 1.85e−05 |
| C22:2 | | | | | |
| C7-DC | Acylcarnitine | 40 | 44 | 0.94 | 2.11e−05 |

For proteins, 38 out of 116 protein concentrations are significantly different between the Test and Control Groups. The 10 proteins with the largest absolute effect between the Test and Control Groups are shown in Table 4.

TABLE 4
Ten Proteins with Largest Effect

| | Measure | Control | ASD | Effect | |
|---|---|---|---|---|---|
| Protein | Category | (n) | (n) | Size | p |
| Retinol-binding | Transport or | 32 | 39 | −1.96 | 9.27e−09 |
| protein 4 | Binding Protein | | | | |
| Thrombospondin-1 | Developmental/Cell | 36 | 33 | −1.67 | 1.91e−06 |
| | Adhesion/Lubrication | | | | |
| | Protein | | | | |
| Coagulation factor | Blood Clotting | 32 | 30 | −1.47 | 2.53e−05 |
| XIII A chain | Protein | | | | |
| Alpha-1-antitrypsin | Miscellaneous | 38 | 38 | −1.18 | 6.90e−06 |
| | Protein | | | | |
| Alpha-2-antiplasmin | Blood Clotting | 20 | 23 | 1.17 | 1.10e−03 |
| | Protein | | | | |
| Apolipoprotein | Transport or | 37 | 40 | 1.15 | 1.61e−05 |
| A-II | Binding Protein | | | | |
| Coagulation | Blood Clotting | 26 | 30 | 1.06 | 2.73e−04 |
| factor X | Protein | | | | |
| Insulin-like growth | Hormone-like | 23 | 29 | −1.02 | 7.22e−04 |
| factor-binding | Protein | | | | |
| protein 2 | | | | | |
| Coagulation | Blood Clotting | 21 | 26 | 0.94 | 4.27e−03 |
| factor XI | Protein | | | | |
| Serotransferrin | Transport or | 38 | 39 | 0.94 | 4.94e−05 |
| | Binding Protein | | | | |

The importance of the analyte contribution is determined using PCA and PLS-DA carried out on the Test and Control Groups and results are compared. According to the present serum metabolomics data, principal component (PC) 1 explained 19.3% of the variation, PC 2 explained 12.7% of the variation and PC 3 explained an additional 8.5%. Together, the first three PCs explained 40.5% of the variation in the metabolomics data. The number of PCs required to explain the total variance (i.e., 100%) is influenced by the number of input variables. Consequently, it is common to have less than 80% of total variance represented in the first three PCs when the input data contains large number of variables, which is typically the case in mass spectrometry-based metabolomics.

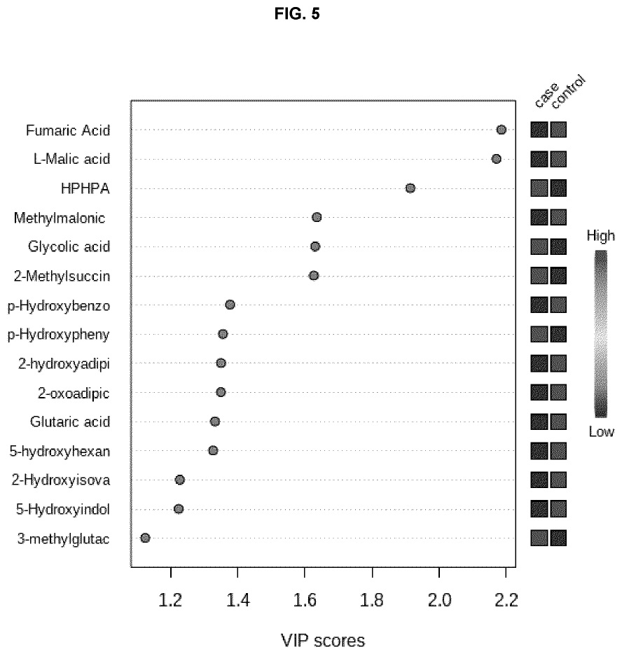

MetaboAnalyst™ is used to generate a PCA plot and start to cluster ASD samples and healthy control samples (see FIG. 6). FIG. 6 shows a score plot of the metabolites analyzed between the Test Group and Control Group. With continued reference to FIG. 6, it is noted that there is separation between the Test and Control Groups, which is replicated in the PLS-DA analysis. In the PLS-DA analysis of serum metabolites, component 1 explains 16.9% of the variation, component 2 explains 9.4% of the variation and component 3 explains an additional 9.1% of the variation.

A PLS-DA plot is generated from the explained variance of the Test Group and Control Group. R2 and Q2 10-fold cross validation values are 0.57 and 0.47, respectively. and also included in FIG. 6. Permutation test confirmed that the separation seen in PLS-DA is statistically significant (p<0.05). As shown in FIG. 6, compared to the PCA results, there is a clearer distinction between the Test Group and Control Group using PLS-DA analysis. Without intending to be limited by theory, it is believed that PLS-DA maximized the covariance between the data (x-variable) the group (y-variable).

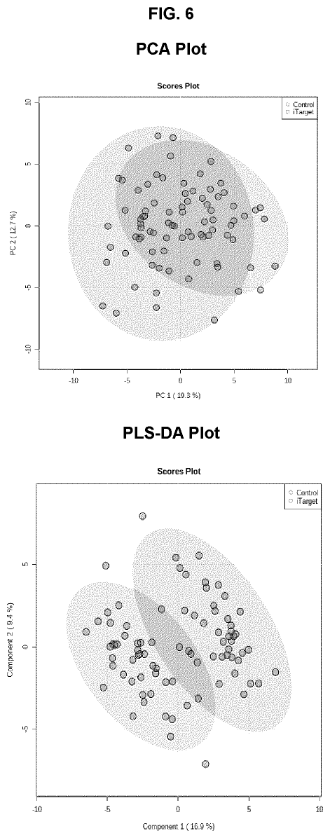

With reference to FIG. 7, a VIP plot is generated to identify the top 20 contributing serum metabolites in descending order where the top variables contribute more to the PLS-DA model than the bottom ones. The importance of each metabolite is calculated using the weighted sum of absolute regression coefficients in the Test and Control Groups and assigned a VIP score.

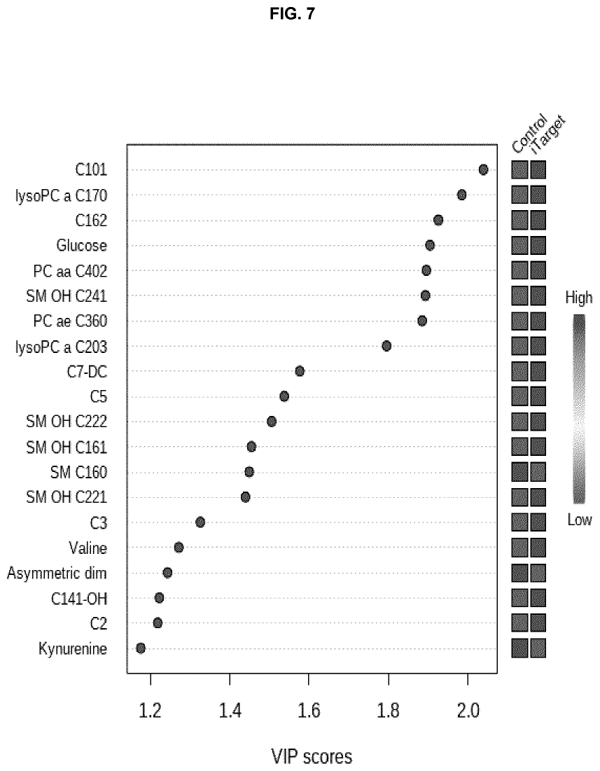

According to the present plasma proteomics data, principal component (PC) 1 explains 18.9% of the variation, PC 2 explains 9.5% of the variation and PC 3 explains 35.1% of the variation. MetaboAnalyst™ is used to generate a PCA plot and start to cluster ASD samples and healthy control samples (see FIG. 8). FIG. 8 shows a score plot of proteomics data analyzed between the Test Group and Control Group. Compared to the serum metabolomics data, there is a larger distinction between the Control and Test Groups for the plasma proteomics analysis.

In the PLS-DA analysis of plasma proteins, component 1 explains 9.6% of the variation, component 2 explains 14.8% of the variation and component 3 explains an additional 8.3% of the variation. A PLS-DA plot is generated from the explained variance of the Test Group and Control Group. A total of 117 proteins are included in the analysis. 10-fold cross validation values are R2=0.72 and Q2=0.69. A permutation test found that the separation distance (B/W) observed is p<0.05, which validates the PLS-CA model. As shown in FIG. 8, compared to the PCA results, there is a clearer distinction between the Test Group and Control Group using PLS-DA analysis.

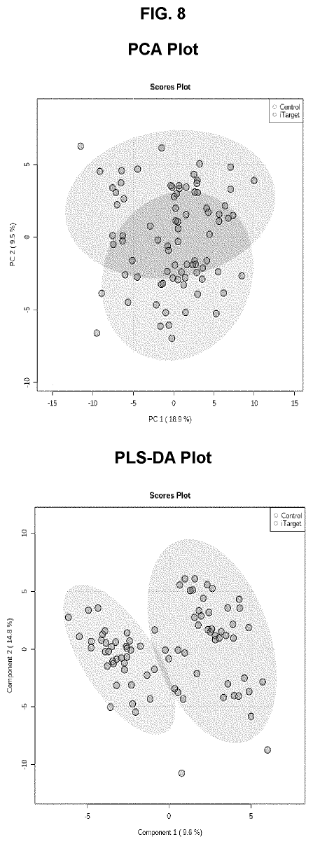

With reference to FIG. 9, a VIP plot is generated to identify the top 20 contributing plasma proteins in descending order where the top variables contribute more to the PLS-DA model than the bottom ones. The importance of each metabolite is calculated using the weighted sum of absolute regression coefficients in the Test and Control Groups and assigned a VIP score.

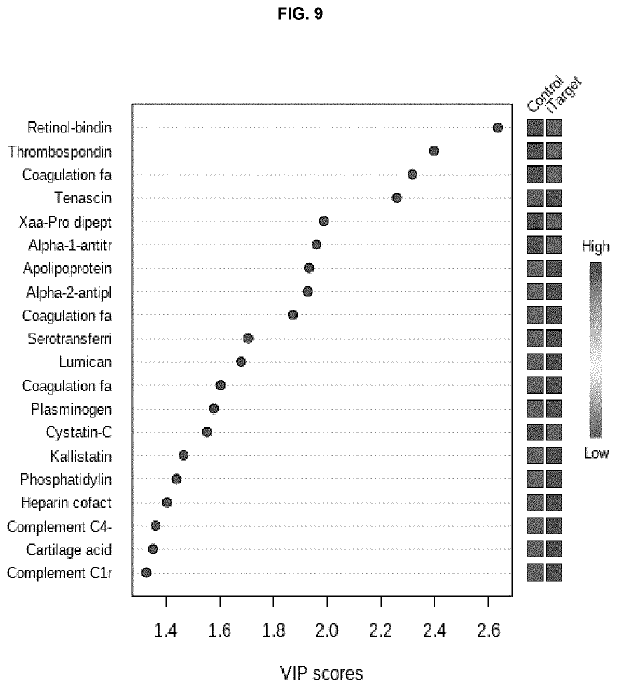

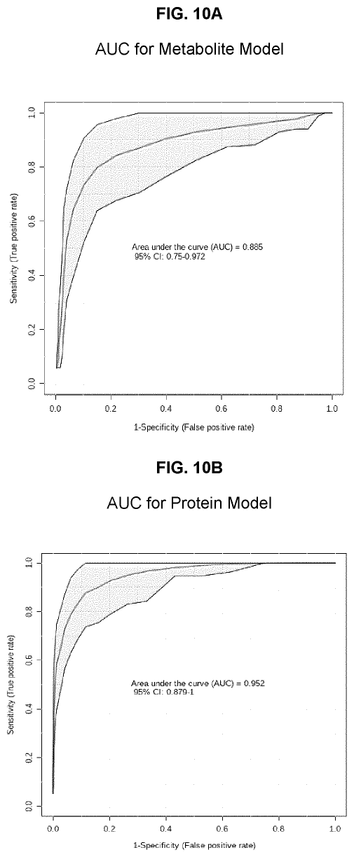

Receiver Operating Characteristic (ROC) curves are generated by Monte-Carlo cross validation (MCCV) using balanced sub-sampling (see FIG. 10A and FIG. 10B). In each MCCV, two thirds of the samples are used to evaluate the importance of the features. The top 5 important features are then used to build classification models which are validated on the ⅓ of the samples that are left out. The classification method used to generate the ROC curve below is based on the results from the PLS-DA using the top 5 serum metabolites (C10:1, C10:1, lysophosphatidylcholine acyl C17:0, C16:2, Glucose, and phosphatidylcholine diacyl C40:2) and the top 5 plasma proteins (Retinol binding protein 4, Thrombospondin, Coagulation factor XIII A chain, Tenascin C, and Xaa-Pro dipeptidase). By computing the area under the ROC curve (AUC), it is possible to determine the diagnostic ability for the metabolite model to discriminate between ASD versus healthy control samples is 0.89. This corresponds to a level of 89% diagnostic accuracy. Similarly, the area under the ROC curve is determined for the protein model to be 0.95, which corresponds to a level of 95% diagnostic accuracy.

Overall, significantly different concentration levels of ASD-related metabolites and ASD-related proteins are identified between serum and plasma samples from ASD and ASD-negative subjects. Notably, there are distinct metabolic and/or proteomic profiles present between the ASD and ASD-negative subjects that are useful for diagnosing ASD.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Elements of the methods and/or systems of the disclosure described in connexion with the examples apply mutatis mutandis to other aspects of the disclosure. Therefore, it goes without saying that the methods and/or systems of the present disclosure encompasses any methods and/or systems comprising any of the steps and/or components cited herein, in any embodiment wherein each such step or component is independently present as defined herein. Many such methods and/or systems than what is specifically set out herein, can be encompassed.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the scope of the present disclosure. It is therefore intended to cover in the appended claims all such changes and modifications that are within the scope of this disclosure.

1. A method for treating Autism Spectrum Disorder (ASD) in a subject in need thereof, the method comprising:
(a) providing a biological sample obtained from the subject;
(b) measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample,
and optionally the acylcarnitine is selected from C10:I, C16:2 and/or C7-DC,
and optionally the lysophospholipid is lysoPC a C17:0, and/or lysoPC a C20:3,
and optionally the sphingolipid is SM (OH) C24:I and/or SM (OH) C22:2,
and optionally the glycerophospholipid is PC ae C36:0 and/or PC aa C40:2;
(c) comparing the concentration levels of the ASD-related metabolites from the obtained sample to the concentration levels of reference ASD-related metabolites from an ASD-negative sample;
(d) identifying the subject as having ASD if the concentration levels of the ASD-related metabolites from the obtained sample are different relative to the concentration levels of the reference ASD-related metabolites from the ASD-negative sample
and optionally the identifying step (d) occurs upon determination that the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the obtained sample are increased relative to the concentration levels of the reference p-hydroxyphenylacetic acid, 2-ethyl-3-hydroxypropionic acid, 3-methylglutaconic acid, 3-hydroxyisovaleric acid, 3-methyl glutaric acid, and/or 4-hydroxyhippuric acid from the ASD-negative sample; and
(e) treating the subject so identified with an ASD treatment regime.

2. The method of claim 1 wherein step
(b) comprises measuring or having measured in a spectroscopy unit the concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample.

3. The method of claim 1, wherein the ASD treatment regime is selected from the croup consisting of: dietary adjustments, nutritional supplements, behaviour training, adjusting the blood levels of one or more of the ASD-related metabolites in the subject diagnosed as having the ASD or predisposed of developing the ASD, and a combination thereof.

4. The method according to claim 3, wherein the adjustment of the blood levels of one or more of the ASD-related metabolites in the subject occurs until an improvement in the behavioural performance in the subject is observed,
and optionally the adjustment of the blood levels of one or more of the ASD-related metabolites comprises adjusting the composition of gut microbiota in the subject.

5. (canceled)

6. The method of claim 1, wherein the identifying step occurs upon determination that the concentration levels of at least one, at least two, at least three, at least four or at least five of the ASD-related metabolites from the obtained sample differ by about 20% or more, about 30% or more, about 40% or more, about 50% or more, about 60% or more, or about 70% or more relative to the concentration levels of the reference ASD-related metabolites from the ASD-negative sample.

7. The method of claim 1, wherein the identifying step (d) occurs upon determination that the concentration levels of the fumaric acid and/or the L-malic acid from the obtained sample are decreased relative to the concentration levels of the reference fumaric acid and/or the reference L-malic acid from the ASD-negative sample, preferably the concentration level of the fumaric acid from the obtained sample is decreased by about 2 times or less relative to the concentration level of the reference fumaric acid from the ASD-negative sample, and/or the concentration level of the L-malic acid from the obtained sample is decreased by about 2 times or less relative to the concentration level of the reference L-malic acid from the ASD-negative sample.

8. The method of claim 1, wherein the identifying step (d) occurs upon determination that the concentration level of the 3-(3-Hydroxyphenyl)-3-hydroxypropanoic acid (HPHPA) from the obtained sample is increased relative to the concentration level of the reference 3-(3-Hydroxyphenyl)-3-hydroxypropanoic acid (HPHPA) from the ASD-negative sample.

9. The method of claim 1, wherein the obtained sample is blood or urine, preferably serum, plasma or urine.

10. The method of claim 2, wherein the spectroscopic technique is selected from the group consisting of liquid chromatography, gas chromatography, liquid chromatography mass spectrometry, gas chromatography mass spectrometry, high performance liquid chromatography mass spectrometry, capillary electrophoresis mass spectrometry, nuclear magnetic resonance spectrometry (NMR), raman spectroscopy, and infrared spectroscopy.

11. The method of claim 1, wherein the comparison of the concentration levels of the ASD-related metabolites from the obtained sample to the concentration levels of the reference ASD-related metabolites from the ASD-negative sample comprises using multivariate statistical analysis.

12. The method according to claim 11, wherein the multivariate statistical analysis is selected from principal component analysis (PCA), or partial least squares projects to latent structures discriminant analysis (PLS-DA).

13. The method of claim 1, wherein the ASD-negative sample is from a non-autistic child aged 10 years or less, 5 years or less, or 3 years or less,
and optionally the subject is a child aged 10 years or less, or 5 years or less, or 3 years or less.

14. (canceled)

15. A method of monitoring Autism Spectrum Disorder (ASD) progression and treating the ASD in a subject in need thereof, the method comprising:
(a) providing a first biological sample obtained from the subject at a first time;
(b) assessing a first ASD-related metabolite profile by measuring concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the first obtained sample;
(c) comparing the first ASD-related metabolite profile with a reference ASD-related metabolite profile from an ASD-negative sample;
(d) determining that there is a first difference between the first ASD-related metabolite profile and the reference ASD-related metabolite profile from the ASD-negative sample, the first difference being indicative of ASD;
(e) providing a second biological sample obtained from the subject at a second time that is after the first time;
(f) assessing a second ASD-related metabolite profile by measuring concentration levels of the ASD-related metabolites from the second obtained sample;
(g) comparing the second ASD-related metabolite profile with the reference ASD-related metabolite profile from the ASD-negative sample;
(h) determining that there is a second difference between the first ASD-related metabolite profile and the reference ASD-related metabolite profile from the ASD-negative sample, the second difference being indicative of ASD;
(i) determining ASD progression based on at least in part on the first and second differences,
and optionally the determining step (i) occurs upon determination that the concentration levels of the 4-hydroxymandelic acid and/or the 2-hydroxyisovaleric acid from the second obtained biological sample are decreased relative to the concentration levels of the 4-hydroxymandelic acid and/or the 2-hydroxyisovaleric acid from the first obtained biological sample; and
(j) treating the subject as identified with an ASD treatment regime.

16. The method according to claim 15, wherein:
(a) the period of time between the first time and the second time is at least 1 month, at least 2 months or at least 3 months;
(b) the first sample, the second sample, or both are blood or urine, preferably both samples are the same specimen type and are selected from serum, plasma or urine;
(c) the acylcarnitine is selected from C10:I, C16:2 and/or C7-DC,
and optionally the identifying step (d) occurs upon determination that the concentration levels of the C10:I, the C16:2 and/or the C7-DC from the obtained sample are increased relative to the concentration levels of the reference C10:I, the reference C16:2 and/or the reference C7-DC from the ASD-negative sample, preferably the concentration levels of the acylcarnitine from the obtained sample is increased by about 2 times or less, preferably from about 0.5 to about 2 times relative to the concentration level of the reference acylcarnitine from the ASD-negative sample,
and optionally the determining step (i) occurs upon determination that the concentration levels of the C10:I, the C16:2 and/or the C7-DC from the second obtained biological sample are increased relative to the concentration levels of the CI 0:1, the C16:2 and/or the C7-DC from the first obtained biological sample;
(d) the determining step (i) occurs upon determination that the concentration levels of the fumaric acid and/or the L-malic acid from the second obtained biological sample are decreased relative to the concentration levels of the fumaric acid and/or the L-malic acid from the first obtained biological sample,
and optionally the determining step (i) occurs upon determination that the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the second obtained biological sample are increased relative to the concentration levels of the p-hydroxyphenylacetic acid, the 2-ethyl-3-hydroxypropionic acid, the 3-methylglutaconic acid, the 3-hydroxyisovaleric acid, the 3-methyl glutaric acid, and/or the 4-hydroxyhippuric acid from the first obtained biological sample;
(e) the determining step (i) occurs upon determination that the concentration level of the 3-(3-Hydroxyphenyl)-3-hydroxypropanoic acid (HPHPA) from the second obtained biological sample is increased relative to the concentration level of the 3-(3-Hydroxyphenyl)-3-hydroxypropanoic acid (HPHPA) from the first obtained biological sample;
(f) the lysophospholipid is lysoPC a C17:0, and/or lysoPC a C20:3;
and optionally the identifying step (d) occurs upon determination that the concentration levels of the lysoPC a C17:0 and/or the lysoPC a C20:3 from the obtained sample are increased relative to the concentration levels of the reference lysoPC a C17:0 and/or the reference lysoPC a C20:3 from the ASD-negative sample, preferably the concentration level of the lysophospholipid from the obtained sample is increased by about 1.1 times or greater, preferably about 1.2 times or greater relative to the concentration level of the reference lysophospholipid from the ASD-negative sample,
and optionally the determining step (i) occurs upon determination that the concentration levels of the lysoPC a C17:0 and/or the lysoPC a C20:3 from the second obtained biological sample are increased related to the concentration levels of the lysoPC a C17:0 and/or the lysoPC a C20:3 from the first obtained biological sample;
(g) the sphingolipid is SM (OH) C24:I and/or SM (OH) C22:2,
and optionally the identifying step (d) occurs upon determination that the concentration levels of the SM (OH) C24:I and/or the SM (OH) C22:2 from the obtained sample are increased relative to the concentration levels of the reference SM (OH) C24:I and/or the reference SM (OH) C22:2 from the ASD-negative sample, preferably the concentration level of the sphingolipid from the obtained sample is increased by about 1.05 times or greater, preferably about 1.1 times or greater relative to the concentration level of the reference sphingolipid from the ASD-negative sample,
and optionally the determining step (i) occurs upon determination that the concentration levels of the SM (OH) C24:1 and/or the SM (OH) C22:2 from the second obtained biological sample SM (OH) C24:I and/or the SM (OH) C22:2 from the obtained sample SM (OH) C24:I and/or the SM (OH) C22:2 from the second obtained biological sample; or
(h) the glycerophospholipid is PC ae C36:0 and/or PC aa C40:2,
and optionally the identifying step (d) occurs upon determination that the concentration levels of the PC ae C36:0 and/or the PC aa C40:2 from the obtained sample are increased relative to the concentration levels of the reference PC ae C36:0 and/or the reference PC aa C40:2 from the ASD-negative sample, preferably the concentration level of the glycerophospholipid from the obtained sample is increased by about 1.05 times or greater, preferably about 1.1 times or greater relative to the concentration level of the reference glycerophospholipid from the ASD-negative sample,
and optionally the determining step (i) occurs upon determination that the concentration levels of the PC ae C36:0 and/or the PC aa C40:2 from the second obtained biological sample are increased relative to the concentration levels of the PC ae C36:0 and/or the PC aa C40:2 from the first obtained biological sample.

17-31. (canceled)

32. The method of claim 1, wherein the identifying step (d) occurs upon determination that the concentration levels of the 4-hydroxymandelic acid and/or the 2-hydroxyisovaleric acid from the obtained sample are decreased relative to the concentration levels of the reference 4-hydroxymandelic acid and/or the reference 2-hydroxyisovaleric acid from the ASD-negative sample, preferably the concentration levels of the 4-hydroxymandelic acid and/or the 2-hydroxyisovaleric acid from the obtained sample are undetectable relative to the concentration levels of the reference 4-hydroxymandelic acid and/or the reference 2-hydroxyisovaleric acid from the ASD-negative sample.

33-35. (canceled)

36. A kit comprising:
(a) reagents for measuring concentration levels of the ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose, optionally together with instructions for use; and/or
(b) (i) a detector configured to detect concentration levels of at least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA) p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from an obtained biological sample;
(ii) a composition comprising fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose in control levels corresponding to a control group of ASD-negative subjects;
(iii) a multivariate analysis system configured to analyze a difference in the concentration levels of the ASD-related metabolites and the control levels, and
and optionally further comprising instructions for an ASD diagnosis method;
wherein the method comprises measuring, using the detector, the levels of the ASD-related metabolites from the obtained biological sample, and comparing the levels of the obtained ASD-related metabolites to the control levels of the ASD-related metabolites obtained from ASD-negative subjects,
and optionally the detector comprises a multi-metabolite detector configured to measure the levels of the ASD-related metabolites comprising the fumaric acid, the L-malic acid, the 4-hydroxymandelic acid, the 2-hydroxyisovaleric acid, the 3-(3-Hydroxyphenyl)-3-hydroxypropanoic acid (HPHPA) p-hydroxyphenylacetic acid, the 2-ethyl-3-hydroxypropionic acid, 3-methylglutaconic acid, the 3-hydroxyisovaleric acid, the 3-methyl glutaric acid, the 4-hydroxyhippuric acid, the acylcarnitine, the lysophospholipid, the sphingolipid, the glycerophospholipid and the glucose.

37-38. (canceled)

39. A computer-implemented method for processing a biological sample of a subject in need thereof, diagnosing an Autism Spectrum Disorder (ASD) and treating the ASD, the computer-implemented method comprising:
(a) receiving a biological sample obtained from the subject in need thereof;
(b) processing the sample in a spectroscopy unit directly or wirelessly linked to a processing device, the processing device having memory for storing measurement data from the spectroscopy unit;
(c) in the spectroscopy unit, measuring levels of least one, at least two, at least three, at least four or at least five ASD-related metabolites selected from the group consisting of fumaric acid, L-malic acid, 4-hydroxymandelic acid, 2-hydroxyisovaleric acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose and storing the measurement data in the processor;
(d) comparing the stored measurement data to a value in the memory representing an ASD-negative sample using multivariate statistical analysis; and
(e) storing on the processing device a result corresponding to at least one, at least two, at least three, at least four or at least five ASD-related metabolites selected from the group consisting of fumaric acid, L-malic acid, 4-hydroxymandelic acid, 2-hydroxyisovaleric
acid, 3-(3-Hydroxyphenyl)-3-hydroxypropanoic acid (HPHPA), p-hydroxyphenylacetic acid, 2-ethyl-3-hydroxypropionic acid, 3-methylglutaconic acid, 3-hydroxyisovaleric acid, 3-methyl glutaric acid, and 4-hydroxyhippuric acid, acylcarnitine, lysophospholipid, sphingolipid, glycerophospholipid and glucose from the obtained sample, wherein the result identifies the subject as having ASD if the measurement data representing the levels of the ASD-related metabolites are different relative to a concentration levels of reference ASD-related metabolites from an ASD-negative sample; and
(f) displaying an ASD treatment regime on an electronic display connected directly or wirelessly to the processor for the subject identified as having ASD or as having predisposition of developing ASD, the displayed treatment regime comprising electronic text on a graphical user interface describing one or more of:
(i) dietary adjustments;
(ii) nutritional supplements;
(iii) behavior training or a combination thereof, to the subject diagnosed as having or predisposed of developing the ASD; or
(iv) adjusting the blood levels of one or more of the ASD-related metabolites in the subject diagnosed as having or predisposed of developing the ASD until an improvement in the behavioral performance in the subject is observed, preferably the adjustment of the blood levels of one or more of the ASD-related metabolites comprises adjusting the composition of gut microbiota in the subject.

40-55. (canceled)
